(19) United States
(12) Patent Application Publication
(10) Pub. No.: US 2023/0357792 A1
(43) Pub. Date: Nov. 9, 2023

(54) METHOD OF ENGINEERING AND ISOLATING ADENO-ASSOCIATED VIRUS

(71) Applicant: Dignity Health, San Francisco, CA (US)

(72) Inventors: Fredric Manfredsson, San Francisco, CA (US); Ivette Sandoval, San Francisco, CA (US)

(21) Appl. No.: 17/906,715

(22) PCT Filed: Mar. 19, 2021
(86) PCT No.: PCT/US21/23314

§ 371 Date: Sep. 19, 2022

Related U.S. Application Data
(60) Provisional application No. 63/141,656, filed on Jan. 26, 2021.
(60) Provisional application No. 62/992,671, filed on Mar. 20, 2020.

(51) Int. Cl. C12N 15/86; C12N 5/0793; G16B 20/30

(52) U.S. Cl. CPC C12N 15/86; C12N 5/0619; G16B 20/30; C12N 2750/14122; C12N 2750/14143

(57) ABSTRACT

Engineered adeno-associated virus (AAV) capsid proteins, each having tropism to a desired target cell or tissue type, are disclosed. Also disclosed are methods of generating the engineered proteins, libraries comprising the engineered proteins, recombinant viruses comprising the engineered proteins, and nucleic acid constructs encoding the engineered proteins.

### CROSS-REFERENCE TO RELATED APPLICATIONS

This application claims priority from Provisional Application No. 62/992,671, filed Mar. 20, 2020, and Provisional Application No. 63/141,656, filed Jan. 26, 2021, the contents of both of which are hereby incorporated by reference in their entirety.

### GOVERNMENTAL RIGHTS

This invention was made with government support under R21AG064221 awarded by the National Institute of Health. The government has certain rights in the invention.

### SEQUENCE LISTING

This application contains a Sequence Listing that has been submitted in ASCII format via EFS-Web and is hereby incorporated by reference in its entirety. The ASCII copy is named 677427_SequenceListing_ST25.txt, and is 647 kilobytes in size.

### FIELD OF THE INVENTION

The present disclosure provides engineered adeno-associated virus (AAV) capsid proteins each having tropism to a desired target cell or tissue type and methods for identifying the engineered capsid AAV proteins from a population of engineered AAV capsid proteins.

### BACKGROUND OF THE INVENTION

Advances in genetic tools have vastly improved our ability to study brain form and function. For instance, the use of inducible transgenic animals, together with other tools such as viral vectors, has allowed for rather intricate experimentation. Nevertheless, a significant barrier still remaining is the lack of tools that allow for very precise manipulations (e.g. overexpression), labeling/inventorying (e.g. cell tracking), targeting, or interrogating (e.g. studying cellular function) of non-neuronal cells such as microglia. For instance, the brain remains one of the true mysteries in science and nature. Although tremendous advances in the last century have shaped our understanding of nervous system function, an in-depth understanding of brain connectivity and cellular interaction is still lacking. This critical need is particularly true for experimentation where multiple distinct populations of cells are being manipulated. Indeed, microglia are becoming increasingly appreciated for performing significant roles important in synaptic formation, maintenance, and pruning. Thus, the ability to be able to perform intricate genetic manipulations of microglia, with the control of parameters such as spatiotemporal control and dosing, and in conjunction with other cell-specific manipulations, is increasingly important

### SUMMARY OF THE INVENTION

One aspect of the present disclosure encompasses a method for identifying from a population of engineered AAV capsid proteins, a capsid protein exhibiting preferential tropism to a desired cell type, such as a cell of microglial lineage. A cell of the desired cell type can be a neural cell. The method comprises generating a plurality of recombinant AAV virions (rAAVs) each comprising an engineered capsid protein encapsidating an AAV vector. The AAV vector has AAV inverted terminal repeats (ITRs) flanking a transgene and an identifier sequence unique to the capsid protein encapsidating the vector. The method further comprises infecting a population of more than one cell type with the generated rAAVs to generate a plurality of transduced cells each comprising a rAAV. The sequence of the unique identifier sequence in each transduced cell and the cell type of each transduced cell are determined to identify the capsid protein present in each cell. The cell type of each cell can comprise determining a transcriptional profile for each cell. A capsid protein exhibiting preferential tropism to the desired cell type is identified based on the presence and absence of the protein in each cell type, wherein the protein exhibits preferential tropism to the desired cell type if the protein is present in the desired cell type and absent in cell types other than the desired cell type. The method can be used to identify a plurality of engineered AAV capsid proteins, each exhibiting preferential tropism to a desired cell type.

In some aspects, the transgene encodes a reporter. When the transgene encodes a reporter, the method can further comprise detecting the transgene in each cell in the population of cells to identify cells transduced with an rAAV.

The engineered protein can comprise a peptide insertion. The peptide insertion can be in a region of the capsid protein of AAV2 selected from I-261, I-381, I-447, I-534, I-573, I-587, I-453, I-520, I-588, I-584, I-585, I-588, I-46, I-115, I-120, I-139, I-161, I-312, I-319, I-459, I-496, I-657, Y257, N258, K259, S391, F392, Y393, C394, Y397, F398, Q536, Q539, or a corresponding position in a capsid protein of another AAV serotype. In some aspects, the engineered capsid protein is an AAV2 capsid protein comprising the Y444F, Y500F, Y730F, T491V, R585S, R588T, R487G amino acid substitutions, or combinations thereof, or corresponding substitutions in the capsid protein of another AAV serotype. In some aspects, the engineered capsid protein is an AAV2 capsid protein comprising the R585S, R588T, and R487G amino acid substitutions, or corresponding substitutions in the capsid protein of another AAV serotype.

Another aspect of the present disclosure encompasses a computerized system for identifying a rAAV exhibiting preferential tropism to a desired cell type. The computerized system comprises a general purpose computer having at least one processor; computer readable memory storing a database of tropism properties exhibited by a plurality of engineered AAV capsid proteins identified using a method as described above; and a computer readable medium comprising functional modules including instructions for the general purpose computer which when executed by the at least one processor, cause the at least one processor to query the database and select among the plurality of engineered AAV capsid proteins a capsid protein exhibiting preferential tropism to a desired cell type.

The database can further comprise a plurality of cell-type-specific transcriptional profile information associated with each cell type, and a plurality of nucleic acid sequences, each sequence encoding a unique engineered AAV capsid protein. The database can further comprises a plurality of identifier sequences, wherein each identifier is unique to a nucleic acid sequence encoding a unique engineered AAV capsid protein.

Yet another aspect of the present disclosure encompasses a recombinant AAV virion (rAAV) library comprising a plurality of rAAV members. Each rAAV member comprises an engineered AAV capsid protein encapsidating an AAV vector, wherein the AAV vector has AAV inverted terminal repeats (ITRs) flanking a transgene and an identifier sequence unique to the capsid protein of each rAAV. Each engineered AAV capsid protein exhibits preferential tropism to a desired cell type. The engineered capsid protein can comprise at least one mutation relative to a wild type capsid protein, and the mutation can be selected from a peptide insertion, an amino acid substitution, and an amino acid deletion. In some aspects, the desired cell type is a glial cell. When the cell is a glial cell, each peptide insertion can be derived from an amino acid sequence of SEQ ID NO 2-183. Each rAAV can exhibit preferential tropism to a desired target cell type.

Additional aspects of the present disclosure encompasses a library of nucleic acid constructs encoding the rAAV library described above, and a plurality of cells comprising the rAAV library, the library of nucleic acid constructs encoding the rAAV library, or a combination thereof.

One aspect of the present disclosure encompasses a method of optimizing delivery of a transgene to a desired cell type in a population of more than one cell type. The method comprises identifying or having identified an engineered AAV capsid protein exhibiting preferential tropism to the desired cell type by the method described above or by the computerized system described above. The method further comprises transducing a population of cells comprising the desired cell type with an rAAV comprising the identified engineered AAV capsid protein to thereby deliver the transgene to the desired cell type. A cell of the desired target cell type can be a central nervous system cell, a microglial cell, or an astrocyte.

An additional aspect of the present disclosure encompasses a kit for identifying or generating engineered AAV capsid proteins exhibiting preferential tropism to a desired target cell type. The kit comprises a library of rAAVs described above, a library of nucleic acid constructs described above, or a plurality of cells comprising the rAAV library, the library of nucleic acid constructs encoding the rAAV library, or a combination thereof.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1A depicts a vector preparation where GFP was controlled by the CAG promoter. Low magnification image showing the area of transduction (mainly neuronal).

FIG. 1B depicts a vector preparation where GFP was controlled by the CAG promoter. In the periphery of the transduced area (possibly because microglial transduction was masked by massive neuronal transduction in the center), numerous transduced (green, GFP) microglia (red; Iba1) can be seen. In order to test the activity of various ubiquitous promoters a separate animal was also injected with a cassette controlled by the CMV promoter (same capsid). Although this image only depicts the transgene (GFP), numerous cells with microglial morphology can be seen throughout. This figure demonstrates that there is no biological “block” of AAV transduction of microglia; moreover, it demonstrates the overall feasibility of our approach.

FIG. 1C depicts a vector preparation where GFP was controlled by the CAG promoter. Confocal imaging of the area outlined in FIG. 1B shows localization of the GFP transgene in microglia.

FIG. 1D depicts a vector preparation where GFP was controlled by the CAG promoter, where a humanized CMV promoter was used.

FIG. 2 Viral library design. The AAV genome is linearized via restriction digestion. Step 1: The viral components are mixed with the microglial ligand (ML) oligonucleotide pool, and barcode (BC) oligonucleotide pool and assembled using a 4-part Gibson assembly in to a genome library (Step 2). The library is duplicated and one copy is subject to CRE recombinase treatment which brings the barcode and ML into close range. This library is subject to paired-end Illumina sequencing, and a database linking the BC to the ML is created (Step 3; arrows indicate sequence primers). Step 4: A second copy of the library is utilized for viral vector generation and in vivo experimentation (Step 5). Step 6: Single cell RNAseq is utilized to identify the profile of infected cells and associated barcodes (arrow indicates RNAseq primer). Genomics is utilized to generate a database where the cellular profile, barcode, and microglial ligand is linked. This database can then be queried to identify capsids that only transduce certain cell-types, in this case microglia.

FIG. 3 Preliminary scRNA seq data. AAV2 was injected into the brain of adult C57bl6 mice. Four days later, animals were sacrificed and nuclei were collected for scRNA seq processing. A preliminary cluster analysis was based on best match to average expression profile of reference transcriptome. Major clusters (neurons, oligodendrocytes and astrocytes, and microglia) were detected. The RNAseq data was also queried for GFP transcripts (i.e. transduced cells) which were found in neurons and oligodendrocytes. Although a cluster of microglial cells was identified, no AAV transcriptional activity was detected in those cells.

FIG. 4A Example of Dual RNAscope ISH/IHC. Section from animal injected with AAV5 was processed for ISH against non-transcribed portion of genome (brown), followed by IHC against IBA1 (blue). Low mag micrograph of the nigra.

FIG. 4B Example of Dual RNAscope ISH/IHC. Section from animal injected with AAV5 was processed for ISH against non-transcribed portion of genome (brown), followed by IHC against IBA1 (blue). High magnification of area outlined in FIG. 4A. With AAV5, no overlap of AAV and Iba1 was found.

FIG. 4C Example of Dual RNAscope ISH/IHC. Section from animal injected with AAV5 was processed for ISH against non-transcribed portion of genome (brown), followed by IHC against IBA1 (blue). High magnification of area outlined in FIG. 4B demonstrating that the microglia is completely void of viral genomes. This also demonstrates the resolution by which the analysis takes place.

FIG. 5 Example of AAV-medicated CRISPR/Cas9 gene inactivation in vivo. A) Surveyor® mutation detection assay showing efficient targeting of the TH gene, determine by the ratio between cleaved (red arrow) and parental (black arrow) DNA fragments. B and C) Representative images of brain sections from AAV-CRISPR treated rats clearly demonstrate robust TH gene knockout in the treated hemisphere (bottom left) which is not seen in the control gRNA treated hemisphere (top left). Similarly, immunofluorescence staining (C) indicates efficient TH (red) gene ablation in cells transduced with AAV-CRISPR-TH (bottom) but not in control brains (top panel). GFP is a marker of AAV-CRISPR transduction. High level of GFP and TH co-localization is obvious in control brains demonstrating lack of toxicity. In contrast, in CRISPR-TH treated brains, the majority of transduced (green) cells show no TH expression (white arrows in high magnification image). D) Quantification of fluorescence intensity (as in indication of TH expression) in AAV-CRISPR transduced cells (GFP+) showing that a majority of cells are homozygous for TH knockout. Herein this workflow is utilized to generate CRISPR specific for EED.

FIG. 5A Example of AAV-medicated CRISPR/Cas9 gene inactivation in vivo. Surveyor® mutation detection assay showing efficient targeting of the TH gene, determine by the ratio between cleaved (red arrow) and parental (black arrow) DNA fragments.

FIG. 5B Example of AAV-medicated CRISPR/Cas9 gene inactivation in vivo. Representative images of brain sections from AAV-CRISPR treated rats clearly demonstrate robust TH gene knockout in the treated hemisphere (bottom left) which is not seen in the control gRNA treated hemisphere (top left).

FIG. 5C Example of AAV-medicated CRISPR/Cas9 gene inactivation in vivo. Immunofluorescence staining indicates efficient TH (red) gene ablation in cells transduced with AAV-CRISPR-TH (bottom) but not in control brains (top panel). GFP is a marker of AAV-CRISPR transduction. High level of GFP and TH co-localization is obvious in control brains demonstrating lack of toxicity. In contrast, in CRISPR-TH treated brains, the majority of transduced (green) cells show no TH expression (white arrows in high magnification image). D) Quantification of fluorescence intensity (as in indication of TH expression) in AAV-CRISPR transduced cells (GFP+) showing that a majority of cells are homozygous for TH knockout. Herein this workflow is utilized to generate CRISPR specific for EED.

FIG. 5C Example of AAV-medicated CRISPR/Cas9 gene inactivation in vivo. Quantification of fluorescence intensity (as in indication of TH expression) in AAV-CRISPR transduced cells (GFP+) showing that a majority of cells are homozygous for TH knockout. Herein this workflow is utilized to generate CRISPR specific for EED.

FIG. 6A Neurolucida based spine quantitation. This figure is aimed to demonstrate feasibility with neuron reconstruction. In this case tissue was Golgi impregnated. The same general approach is taken in other experiments albeit using YFP instead of Golgi.

FIG. 6 Neurolucida based spine quantitation. This figure is aimed to demonstrate feasibility with neuron reconstruction. Striatal medium spiny neuron was reconstructed using Neurolucida and optical sectioning. The same general approach is taken in other experiments albeit using YFP instead of Golgi.

FIG. 7A. In vivo recording of striatal MSN single activity. Inset shows traces of cortical-evoked striatal activity at increasing intensities of stimulation (arrows: stimulus artifact).

FIG. 7B. Spontaneous activity of a MSN recorded from a sham control rat.

FIG. 7C. Typical MSN response (same cell as in FIG. 7B) to somatic current injections. Red arrow heads are the onset-offset artifacts of the current-induced juxtacellular ejection.

FIG. 8. Photograph of spinal cord harvested from Sprague Dawley rats injected with an rAAV2 having improved neuronal tropism.

FIG. 9. Photograph of immunostaining of sections of the spinal cord in FIG. 8.

FIG. 10. The first panel is a photograph of immunostaining of an L3 section of the spinal cord in FIG. 8. The second panel is a photograph taken at higher magnification of the L3 section, showing transgene expression in both dorsal and ventral horns of the spinal cord.

FIG. 11. Upper panels; photographs of immunostaining and FISH staining of a section of the spinal cord in FIG. 8. Lower panels; results from a 1:10 dilution of the vector (negative control).

FIG. 12. Higher magnification of the section of immunostaining and FISH staining of the spinal cord of FIG. 11.

FIG. 13. Higher magnification of the section of FISH staining of sections of the spinal cord of FIG. 11 at the dorsal horn, intermediate zone, and ventral horn.

FIG. 14. Photographs of immunostaining of sections of a spinal cord injected with a GFP-expressing rAAV2.

FIG. 15. Sagittal section from animal receiving injection as outlined in FIG. 8 (upper panel) and a photograph of the motor cortex taken at higher magnification

### DETAILED DESCRIPTION

The present disclosure is based in part on the development of a method for identifying an adeno-associated virus (AAV) capsid protein exhibiting tropism to a desired cell type from a population of engineered AAV capsid proteins. Methods described herein can be used to identify engineered capsid proteins having the ability to target cell types not normally targeted by the wild type version of the protein. In addition, the methods described herein can be used to identify engineered capsid proteins exhibiting preferential tropism to a desired cell type. Methods described can also be used to generate libraries of recombinant AAV virions (rAAVs) and nucleic acids encoding the libraries of rAAVs, wherein each engineered AAV capsid protein exhibits tropism to a select cell type. The disclosure also provides computerized systems configured to perform in silico selection to identify rAAVs exhibiting tropism to a desired cell type. Other aspects of the present disclosure are also described.

### I. Method of Identifying AAV Capsid Proteins

One aspect of the present disclosure encompasses a method for identifying from a population of engineered AAV capsid proteins, a capsid protein exhibiting preferential tropism to a desired cell type. Engineered capsid proteins can be as described in Section I(b) below.

The method comprises generating a plurality of recombinant AAV virions (rAAVs) each comprising an engineered AAV capsid protein encapsidating an AAV vector. For instance, 1, 2, 3, 4, 5, 6, 7, 8, 9, 10, or a library of rAAVs, can be generated, wherein each rAAV comprises an engineered AAV capsid protein exhibiting preferential tropism to a desired cell type. Libraries of rAAVs can be as described in Section II below. A population of cells of more than one cell type are infected with the rAAVs to generate a plurality of transduced cells each comprising an rAAV. Cells and cell types can be as described in Section I(c) below, and methods of infecting cells can be as described in Section I(d) below.

The sequence of the unique identifier in each transduced cell, and the cell type of a cell comprising the unique identifier are determined, thereby matching each unique identifier with the cell type of the cell comprising the identifier. As further explained in Section II below, each unique identifier is matched to one engineered capsid protein. Accordingly, determining the sequence of an identifier in a cell transduced with a rAAV comprising an engineered capsid protein encapsidating a vector comprising the identifier, also identifies the matched engineered capsid protein in the transduced cell.

The ability to identify a cell type that an rAAV can transduce, provides the capability to identify engineered capsid proteins capable of transducing cell types normally refractory to infection by wild types of AAV capsid proteins. For instance, the inventors were able to identify at least one engineered capsid protein with altered tropism capable of transducing microglia, a cell type remarkably refractory to infection by wild types of AAV capsid proteins.

Importantly, it is also possible to determine each cell type that a certain engineered capsid protein cannot transduce. Accordingly, positive and negative selection can be used to determine the preferential tropism exhibited by an engineered capsid protein to a cell type in a population of more than one cell type. More specifically, a capsid protein exhibiting preferential tropism to a desired cell type can be identified based on the presence and absence of the protein in each cell type, wherein the protein exhibits preferential tropism to the desired cell type if the protein is present in the desired cell type and absent in cell types other than the desired cell type.

### (a) Adeno Associated Virus (AAV)

A rAAV of the instant disclosure comprises an AAV capsid protein encapsidating an AAV vector. Briefly, AAV vectors generally comprise the AAV inverted terminal repeats (ITRs) of the virus flanking heterologous nucleic acid sequences of interest. AAV ITRs contain all cis-acting elements involved in AAV genome rescue, replication, and packaging. Accordingly, the ITRs can be segregated from the viral encoding regions allowing for AAV vector design that comprises only the ITRs of the virus flanking heterologous nucleic acid sequences of interest. Generally, rAAV particles are generated by transfecting producer cells with a plasmid (AAV cis-plasmid) containing a cloned AAV vector, and a separate construct expressing in trans the viral rep and cap genes. The adenovirus helper factors, such as E1A, E1B, E2A, E4ORF6 and VA RNAs, can be provided by either adenovirus infection or transfecting into production cells a third plasmid that provides these adenovirus helper factors.

An AAV vector of the instant disclosure comprises AAV ITRs flanking a transgene and an identifier nucleic acid sequence (also referred to herein as identifier sequence, unique identifier or simply identifier) unique to the capsid protein of each rAAV. In some aspects, the transgene may also encode a reporter. As used herein, a “transgene” refers to any nucleic acid molecule, e.g., a DNA molecule having a nucleic acid sequence foreign to a cell to which the molecule is introduced. For example, the DNA molecule may have a nucleic acid sequence encoding a protein of interest which is foreign to the cell to which the DNA molecule is introduced. Expression of the reporter can be used to determine transduction efficiency of the rAAV and identify cells successfully transduced by the rAAV during experimentation. Reporters can be as described in Section I(d) herein below.

As further explained in Section II below, each unique identifier is matched to one engineered protein and is used to identify the matched engineered capsid protein in transduced cell. In some aspects, an AAV vector further comprises at least one second identifier. The second identifier can, for example, be used to identify a library of rAAVs. Accordingly, when a second identifier is used to identify a library of rAAVs, the second identifier has a sequence in common with all second identifiers in the library of AAVs.

An AAV as described herein can be any AAV serotype, including a serotype selected from AAV1, AAV2, AAV3, AAV4, AAV5, AAV6, AAV7, AAV8, AAVrh8, AAVrh10, AAVrh39, or AAVrh43. The rAAVs of the disclosure can be pseudotyped rAAVs. Pseudotyping is the process of producing viruses or viral vectors in combination with foreign viral envelope proteins. The result is a pseudotyped virus particle comprising a vector derived from AAV serotype encapsidated by an AAV capsid protein from a different serotype. Accordingly, the foreign viral envelope proteins can be used to alter host tropism or an increased/decreased stability of the virus particles. In some aspects, a pseudotyped rAAV comprises nucleic acids from two or more different AAVs, wherein the nucleic acid from one AAV encodes a capsid protein, and the nucleic acid of at least one other AAV encodes other viral proteins and/or the viral genome. For example, a pseudotyped AAV vector containing the ITRs of serotype X encapsidated with the proteins of Y is designated as AAVX/Y (e.g., AAV2/1 has the ITRs of AAV2 and the capsid of AAV1). In some aspects, the AAV serotype is AAV2.

In some aspects, the capsid protein is an AAV2 capsid protein. The AAV capsid protein can be a capsid protein of AAV2 having an amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to the amino acid sequence of SEQ ID NO: 1. In some aspects, the capsid protein is a capsid protein of AAV2 having an amino acid sequence at least 85%, at least 90%, at least 95%, or at least 99% identical to the amino acid sequence of SEQ ID NO: 1.

### (b) Engineered AAV Capsid Proteins

Methods of the instant disclosure can identify an engineered AAV capsid protein exhibiting preferential tropism to a desired cell type. As used herein, the term “preferential tropism” or “preferentially tropic” when applied to an engineered AAV capsid protein, can be used interchangeably and refer to the ability of recombinant AAV virions (rAAVs) comprising the engineered protein to transduce a desired cell type over cell types other than the desired cell types in a population of cells comprising more than one cell type. Accordingly, an engineered AAV capsid protein said to be preferentially tropic to a desired cell type exhibits a higher transduction efficiency in the desired cell type when compared to the transduction efficiency in cell types other than the desired cell type. For instance, an engineered protein exhibiting tropism to a desired cell type can have a 2, 3, 4, 5, 6, 7, 8, 9, 10, 20, 30, 40, 50, 100, 200, 300, 400, 500, or more times higher transduction efficiency in the desired cell type when compared to the transduction efficiency of the engineered protein in cell types other than the desired cell type. The transduction efficiency of a given AAV capsid protein is determined by the efficiency of each of the different steps in the AAV life cycle. Methods of determining transduction efficiency of a rAAV are known and include measuring the level of expression of a transgene of the rAAV in cells infected with the rAAV.

An engineered capsid protein comprises one or more mutations relative to a wild type capsid protein. A mutation can be a peptide insertion, an amino acid substitution, or an amino acid deletion. An engineered capsid protein can also be a chimeric capsid protein comprising fragments of capsid proteins of various AAV serotypes.

In some aspects, the engineered AAV capsid protein comprises one or more peptide insertions in the capsid protein. In some aspects, the engineered AAV capsid protein comprises two or more peptide insertions in the capsid protein. A peptide can be any sequence of sufficient length to modify the tropism of an engineered capsid protein.

For instance, a peptide can be about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 50, 55, 60, 65, 70, 75, 80, 85, 90, 95 amino acids in length or longer. A peptide can also be about 5-10, 7-15, 10-15, 10-20, 15-20, 20-25, 20-30, 30-35, 30-40, 35-40, 40-45, 40-50, 45-50, 50-55, 50-60, 55-60, 60-65, 60-70, 65-70, 70-75, 70-80, 75-80, 80-85, 80-90, 85-90, 90-95, 90-100, 95-100, or more than 100 amino acids in length or any individual length within these ranges.

In some aspects, an insertion is in one or more surface exposed loop regions of the capsid protein. In some aspects, the insertion is in one or more variable regions of the capsid protein. The one or more insertion site can be in a region of the capsid protein of AAV2 selected from I-261, I-381, I-447, I-534, I-573, I-587, I-453, I-520, I-588, I-584, I-585, I-588, I-46, I-115, I-120, I-139, I-161, I-312, I-319, I-459, I-496, I-657 or a corresponding position in a capsid protein of another AAV serotype.

Disruption of the surface exposed loop regions such as by insertion of a peptide in the loop region, also disrupts canonical entry of AAV mediated via HSPG binding, a secondary receptor of AAV. Disrupting AAV entry mediated via HSPG binding can enhance binding to the AAVR receptor, thereby improving transduction efficiency of a desired cell type. The inventors also surprisingly discovered that modifying the following amino acid residues outside the surface exposed loop regions can also disrupt entry mediated via HSPG binding and improve transduction efficiency of a desired cell type: S153, D169, T174, D176, D177, or K178, or combinations thereof. Further, the inventors discovered the following amino acid residues outside the surface exposed loop regions that, when modified, can enhance binding to the AAVR receptor and improve transduction efficiency of a desired cell type: Y257, N258, K259, S391, F392, Y393, C394, Y397, F398, Q536, Q539, or combinations thereof. Accordingly engineered capsid proteins of the instant disclosure can have a mutation in the surface exposed loop region, at the S153, D169, T174, D176, D177, K178, Y257, N258, K259, S391, F392, Y393, C394, Y397, F398, Q536, Q539 amino acid residues of the AAV2 capsid protein, or combinations thereof, or corresponding substitutions in the capsid protein of another AAV serotype to enhance binding to the AAVR receptor. Engineered capsid proteins comprising these mutations can be used as a starting sequence to generate engineered capsid proteins having preferential tropism using methods of the instant disclosure. In some aspects, engineered capsid proteins of the instant disclosure comprise the Y444F, Y500F, Y730F, T491V, R585S, R588T, R487G mutations, or combinations thereof. In some aspects, engineered capsid proteins of the instant disclosure comprise the R585S, R588T, and R487G mutations. This engineered capsid protein exhibits considerably improved transduction efficiency, and can be used as a starting sequence to generate engineered capsid proteins having preferential tropism using methods of the instant disclosure.

In some aspects, a peptide inserted into the capsid protein is a ligand of a cell type of interest. In other aspects, a peptide inserted into the capsid protein is derived from a ligand of a cell type of interest. A peptide derived from a ligand can be a mutated ligand, a fragment of the ligand, or a mutated fragment of a ligand of a cell type of interest. For instance, a peptide derived from a ligand can have an amino acid sequence at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical to a mutated ligand, a fragment of the ligand, or a mutated fragment of a ligand.

When the cell of the desired cell type is of glial lineage, a peptide can be an amino acid sequence selected from SEQ ID NO 2-183 or a peptide derived therefrom. In some aspects, the peptide is an amino acid sequence selected from SEQ ID NO 2-153 or a peptide derived therefrom. In other aspects, the peptide is an amino acid sequence selected from SEQ ID NO 154-176 or a peptide derived therefrom. In yet other aspects, the peptide is an amino acid sequence selected from SEQ ID NO 177-183 or a peptide derived therefrom.

In one aspect, the peptide is an amino acid sequence having 85% or more identity with SEQ ID NO 154 or a peptide derived therefrom. In one aspect, the peptide is an amino acid sequence having 85% or more identity with SEQ ID NO 155 or a peptide derived therefrom. In one aspect, the peptide is an amino acid sequence having 85% or more identity with SEQ ID NO 156 or a peptide derived therefrom. In one aspect, the peptide is an amino acid sequence having 85% or more identity with SEQ ID NO 157 or a peptide derived therefrom. In one aspect, the peptide is an amino acid sequence having 85% or more identity with SEQ ID NO 158 or a peptide derived therefrom. In one aspect, the peptide is an amino acid sequence having 85% or more identity with SEQ ID NO 159 or a peptide derived therefrom. In one aspect, the peptide is an amino acid sequence having 85% or more identity with SEQ ID NO 160 or a peptide derived therefrom. In one aspect, the peptide is an amino acid sequence having 85% or more identity with SEQ ID NO 161 or a peptide derived therefrom. In one aspect, the peptide is an amino acid sequence having 85% or more identity with SEQ ID NO 162 or a peptide derived therefrom. In one aspect, the peptide is an amino acid sequence having 85% or more identity with SEQ ID NO 163 or a peptide derived therefrom. In one aspect, the peptide is an amino acid sequence having 85% or more identity with SEQ ID NO 164 or a peptide derived therefrom. In one aspect, the peptide is an amino acid sequence having 85% or more identity with SEQ ID NO 165 or a peptide derived therefrom. In one aspect, the peptide is an amino acid sequence having 85% or more identity with SEQ ID NO 166 or a peptide derived therefrom. In one aspect, the peptide is an amino acid sequence having 85% or more identity with SEQ ID NO 167 or a peptide derived therefrom. In one aspect, the peptide is an amino acid sequence having 85% or more identity with SEQ ID NO 168 or a peptide derived therefrom. In one aspect, the peptide is an amino acid sequence having 85% or more identity with SEQ ID NO 169 or a peptide derived therefrom. In one aspect, the peptide is an amino acid sequence having 85% or more identity with SEQ ID NO 170 or a peptide derived therefrom. In one aspect, the peptide is an amino acid sequence having 85% or more identity with SEQ ID NO 171 or a peptide derived therefrom. In one aspect, the peptide is an amino acid sequence having 85% or more identity with SEQ ID NO 172 or a peptide derived therefrom. In one aspect, the peptide is an amino acid sequence having 85% or more identity with SEQ ID NO 173 or a peptide derived therefrom. In one aspect, the peptide is an amino acid sequence having 85% or more identity with SEQ ID NO 174 or a peptide derived therefrom. In one aspect, the peptide is an amino acid sequence having 85% or more identity with SEQ ID NO 175 or a peptide derived therefrom. In one aspect, the peptide is an amino acid sequence having 85% or more identity with SEQ ID NO 176 or a peptide derived therefrom. In one aspect, the peptide is an amino acid sequence having 85% or more identity with SEQ ID NO 177 or a peptide derived therefrom. In one aspect, the peptide is an amino acid sequence having 85% or more identity with SEQ ID NO 178 or a peptide derived therefrom. In one aspect, the peptide is an amino acid sequence having 85% or more identity with SEQ ID NO 179 or a peptide derived therefrom. In one aspect, the peptide is an amino acid sequence having 85% or more identity with SEQ ID NO 180 or a peptide derived therefrom. In one aspect, the peptide is an amino acid sequence having 85% or more identity with SEQ ID NO 181 or a peptide derived therefrom. In one aspect, the peptide is an amino acid sequence having 85% or more identity with SEQ ID NO 182 or a peptide derived therefrom. In one aspect, the peptide is an amino acid sequence having 85% or more identity with SEQ ID NO 183 or a peptide derived therefrom.

### (c) Cell Type

A method of the instant disclosure comprises determining the cell type of each infected cell. Cell type can be determined using methods know in the art. Non-limiting examples of methods used for determining cell type include the identification of cell markers and determining a transcriptional profile of a cell. In some aspects, the cell type is determined by identifying cell markers that distinguish unique cell types. Cell markers can be expressed both extracellularly on the cells surface or as an intracellular molecule. In other aspects, the cell type is determined by determining a transcriptional profile of a cell. The transcriptional profile of a cell can be determined using single cell sequencing of RNA transcripts (scRNA-seq). Standard methods such as microarrays and bulk RNA-seq analysis analyze the expression of RNAs from large populations of cells.

An engineered capsid protein of the instant disclosure exhibits preferential tropism to a desired cell type. The desired cell type can be an epithelial cell, a cell in an organ in the body, a cell in connective tissue, muscle tissue, and nervous tissue including the central nervous system and the peripheral nervous system, circulatory system, a cancer cell or tumor, or a cell of the immune system. In some aspects, the desired cell type is a cell in the central nervous system. Non-limiting examples of cell types in the nervous system include axons, oligodendrocytes, neuroblasts, neurons, glial cells, and astrocytes. In some aspects, the desired cell type is a cell of glial lineage. In some aspects, the desired cell type is a microglial cell. In other aspects, the desired cell type is an astrocyte.

In some aspects, the desired cell type is a cancer cell. The cancer cell can be a glioblastoma, colon cancer, ovarian cancer, breast cancer, prostate cancer, osteosarcoma, or malignant melanoma. Other non-limiting examples of neoplasms or cancer cells that may be suitable for use in methods of the instant disclosure include acute lymphoblastic leukemia, acute myeloid leukemia, adrenocortical carcinoma, AIDS-related cancers, AIDS-related lymphoma, anal cancer, appendix cancer, astrocytomas (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, ependymoma, medulloblastoma, supratentorial primitive neuroectodermal tumors, visual pathway and hypothalamic gliomas), breast cancer, bronchial adenomas/carcinoids, Burkitt lymphoma, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor (childhood), extragonadal germ cell tumor, extrahepatic bile duct cancer, eye cancers (intraocular melanoma, retinoblastoma), gallbladder cancer, gastric (stomach) cancer, gastrointestinal carcinoid tumor, gastrointestinal stromal tumor, germ cell tumors (childhood extracranial, extragonadal, ovarian), gestational trophoblastic tumor, gliomas (adult, childhood brain stem, childhood cerebral astrocytoma, childhood visual pathway and hypothalamic), gastric carcinoid, hairy cell leukemia, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenström), malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, myeloproliferative disorders (chronic), nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer (islet cell), paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma (childhood), salivary gland cancer, sarcoma (Ewing family of tumors, Kaposi, soft tissue, uterine), Sézary syndrome, skin cancers (nonmelanoma, melanoma), skin carcinoma (Merkel cell), small cell lung cancer, small intestine cancer, soft tissue sarcoma, squamous cell carcinoma, squamous neck cancer with occult primary (metastatic), stomach cancer, supratentorial primitive neuroectodermal tumor (childhood), T-Cell lymphoma (cutaneous), testicular cancer, throat cancer, thymoma (childhood), thymoma and thymic carcinoma, thyroid cancer, thyroid cancer (childhood), transitional cell cancer of the renal pelvis and ureter, trophoblastic tumor (gestational), unknown primary site (adult, childhood), ureter and renal pelvis transitional cell cancer, urethral cancer, uterine cancer (endometrial), uterine sarcoma, vaginal cancer, visual pathway and hypothalamic glioma (childhood), vulvar cancer, Waldenström macroglobulinemia, and Wilms tumor (childhood).

In some aspects, the desired cell type is an immune cell such as a lymphocytes, neutrophils, microglia, and monocytes/macrophages, or combinations thereof. In some aspects, the target cell or tissue type is monocytes or microglia.

### (d) Cell Infection

Cell infection methods for infecting cells with the rAAVs are known. For instance, the cells can be infected with the rAAVs by contacting the cells with the rAAVs. For instance, the cells can be tissue culture cells, and they can be contacted with the rAAVs by adding the rAAVs to the cell culture. The cells can also be infected by delivering to a subject in compositions according to any appropriate methods known in the art. The rAAV, preferably suspended in a physiologically compatible carrier (e.g., in a composition), may be administered to a subject, e.g., host animal, such as a human, mouse, rat, cat, dog, sheep, rabbit, horse, cow, goat, pig, guinea pig, hamster, chicken, turkey, or a non-human primate (e.g., Macaque).

Delivery of the rAAVs to a mammalian subject may be by, for example, intramuscular injection or by administration into the bloodstream of the mammalian subject. Administration into the bloodstream may be by injection into a vein, an artery, or any other vascular conduit. In some aspects, the rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique can also be employed by the skilled artisan to administer the virions into the vasculature of an isolated limb to potentially enhance transduction into muscle cells or tissue. Moreover, in certain aspects, it may be desirable to deliver the virions to the CNS of a subject. By “CNS” is meant all cells and tissue of the brain and spinal cord of a vertebrate. Thus, the term includes, but is not limited to, neuronal cells, glial cells, astrocytes, cerebrospinal fluid (CSF), interstitial spaces, bone, cartilage and the like. Recombinant AAVs may be delivered directly to the CNS or brain by injection into, e.g., the ventricular region, as well as to the striatum (e.g., the caudate nucleus or putamen of the striatum), spinal cord and neuromuscular junction, or cerebellar lobule, with a needle, catheter or related device, using neurosurgical techniques known in the art, such as by stereotactic injection.

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the disclosure.

Optionally, in addition to the rAAV and carrier(s), other conventional pharmaceutical ingredients can be included, such as preservatives or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

rAAVs are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., intraportal delivery to the liver), oral, inhalation (including intranasal and intratracheal delivery), intraocular, intravenous, intramuscular, subcutaneous, intradermal, intratumoral, and other parental routes of administration. Routes of administration may be combined, if desired.

In some aspects, rAAV compositions are formulated to reduce aggregation of AAV particles in the composition, particularly where high rAAV concentrations are present (e.g., ⁻10¹³ GC/ml or more). Methods for reducing aggregation of rAAVs are well known in the art and include, for example, addition of surfactants, pH adjustment, salt concentration adjustment, etc.

Formulation of pharmaceutically-acceptable excipients and carrier solutions is well known to those of skill in the art, as is the development of suitable dosing and treatment regimens for using the particular compositions described herein in a variety of treatment regimens. Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically-useful composition may be prepared in such a way that a suitable dosage is obtained in any given unit dose of the compound. Factors such as solubility, bioavailability, biological half-life, route of administration, product shelf life, as well as other pharmacological considerations, are contemplated by one skilled in the art of preparing such pharmaceutical formulations, and as such, a variety of dosages and treatment regimens may be desirable.

In certain aspects, it is desirable to deliver the rAAV-based therapeutic constructs in suitably formulated pharmaceutical compositions disclosed herein either subcutaneously, intrapancreatically, intranasally, parenterally, intravenously, intramuscularly, intrathecally, or orally, intraperitoneally, or by inhalation. In some aspects, the cells are infected with the rAAVs by administering the rAAVs to a subject in a pharmaceutically-acceptable carrier to the subject in an amount and for a period of time sufficient to infect the cells. For instance, the rAAVs can be administered parenterally into the subject. When the cells are neural cells, including microglial cells, the rAAVs can be administered by injection into the striatum.

Pharmaceutical forms suitable for injectable use can include sterile aqueous solutions or dispersions and sterile powders for the extemporaneous preparation of sterile injectable solutions or dispersions. Dispersions may also be prepared in glycerol, liquid polyethylene glycols and mixtures thereof, and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it is preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed is known to those of skill in the art. For example, one dosage may be dissolved in 1 ml of isotonic NaCl solution and either added to 1000 ml of hypodermoclysis fluid or injected at the proposed site of infusion. Some variation in dosage may necessarily occur depending on the condition of the host.

Sterile injectable solutions can be prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient, plus any additional desired ingredient from a previously sterile-filtered solution thereof.

rAAVs can also be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions are administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the disclosure into suitable host cells. In particular, the rAAV vector delivered transgenes may be formulated for delivery either encapsulated in a lipid particle, a liposome, a vesicle, a nanosphere, or a nanoparticle or the like.

### (e) Reporters

The transgene may also include a nucleic acid sequence encoding a reporter molecule. As used herein, the term “reporter” refers to any biomolecule that may be used as an indicator of transcription and/or translation through a promoter. A reporter may be a polypeptide. A reporter may also be a nucleic acid. Suitable polypeptide and nucleic acid reporters are known in the art, and may include visual reporters, selectable reporters, screenable reporters, and combinations thereof. Other types of reporters will be recognized by individuals of skill in the art.

Visual reporters typically result in a visual signal, such as a color change in the cell, or fluorescence or luminescence of the cell. Suitable visual reporters include fluorescent proteins, visible reporters, epitope tags, affinity tags, RNA aptamers, and the like. Non-limiting examples of suitable fluorescent proteins include green fluorescent proteins (e.g., GFP, GFP-2, tagGFP, turboGFP, EGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellow1), blue fluorescent proteins (e.g., EBFP, EBFP2, Azurite, mKalama1, GFPuv, Sapphire, T-sapphire), cyan fluorescent proteins (e.g., ECFP, Cerulean, CyPet, AmCyan1, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (e.g., mOrange, mKO, Kusabira-Orange, Monomeric Kusabira-Orange, mTangerine, tdTomato), or any other suitable fluorescent protein. Non-limiting examples of visual reporters include luciferase, alkaline phosphatase, beta-glucuronidase (GUS), beta-galactosidase, beta-lactamase, horseradish peroxidase, anthocyanin pigmentation, and variants thereof. Suitable epitope tags include, but are not limited to, myc, AcV5, AU1, AU5, E, ECS, E2, FLAG, HA, Maltose binding protein, nus, Softag 1, Softag 3, Strep, SBP, Glu-Glu, HSV, KT3, S, S1, T7, V5, VSV-G, 6×His, BCCP, and calmodulin. Non-limiting examples of affinity tags include chitin binding protein (CBP), thioredoxin (TRX), poly(NANP), tandem affinity purification (TAP) tag, and glutathione-S-transferase (GST). Non-limiting examples of RNA aptamers include fluorescent RNA aptamers that sequester small molecule dyes and activate their fluorescence.

Other visual reporters may include fluorescent resonance energy transfer (FRET), lanthamide resonance energy transfer (LRET), fluorescence cross-correlation spectroscopy, fluorescence quenching, fluorescence polarization, scintillation proximity, chemiluminescence energy transfer, bioluminescence resonance energy transfer, excimer formation, phosphorescence, electrochemical changes, molecular beacons, and redox potential changes.

It will be recognized that combinations of reporters may be used. For instance, a visual reporter fused to a protein expressed by the gene of interest may be used to identify an accurate homologous recombination event, but the visual reporter is not permanently fused to the protein. A second reporter may be used in combination with the visual reporter, wherein the second reporter is permanently fused to the protein.

Additionally, irrespective of the reporter used in a transgene, the reporter may be a split reporter system. Split reporter systems may be used to reduce the size of a reporter sequence in a transgene. Non-limiting examples of suitable split reporter systems include split GFP systems, split 5-EnolpyruvylShikimate-3-Phosphate Synthase for glyphosate resistance, among others. Similarly, irrespective of the reporter used, a transgene may encode an activator for activating a reporter encoded in a location other than the AAV vector.

II. rAAV Libraries

The present disclosure also encompasses a library of rAAVs comprising a plurality of rAAV members. Each rAAV member comprises an engineered AAV capsid protein encapsidating an AAV vector, wherein the AAV vector has AAV inverted terminal repeats (ITRs) flanking a transgene and an identifier sequence unique to the capsid protein of each rAAV. Each engineered AAV capsid protein exhibits preferential tropism to a select cell type. The number of rAAVs in a library can and will differ depending on the number of engineered capsid proteins and the population of cells to be infected by rAAV members of the library among other variables.

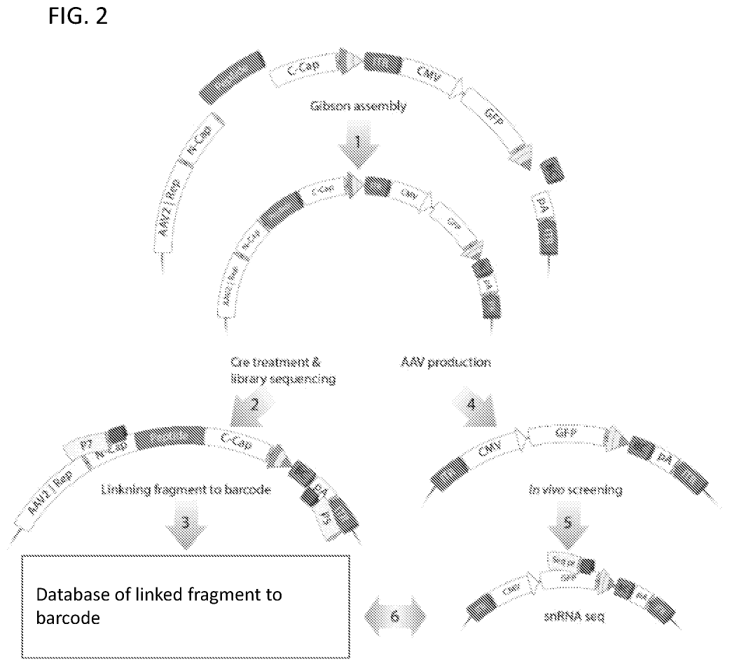

A viral library of the instant disclosure can be generated as described in Example 2 and outlined in FIG. 2 of the instant disclosure. In short, a cloning plasmid is constructed containing the following key features: 1) AAV genes housed outside the ITRs ensuring that serial infectivity cannot occur during virus production. 2) Unidirectional LoxP sites are introduced immediately downstream of the AAV cap gene encoding a genetically engineered capsid protein and immediately upstream of a unique identifier. The cloning plasmid is constructed using methodology designed to maximize diversity whilst retaining complete unambiguity (i.e. 1 identifier=1 capsid variant), and to ensure fidelity of barcode/microglial ligand sequencing (Example 2).

The library is duplicated and one copy is subjected to CRE recombinase treatment which brings the barcode and ML into close range. Upon CRE-mediated recombination, a fragment is excised, bringing the identifier and engineered mutations in the capsid protein in close proximity. This facilitates sequencing of the identifier together with the ligand sequence in order to build a reference database having each identifier matched with one engineered capsid protein, or a mutation in the engineered capsid protein that confers preferential tropism to the engineered protein.

A second copy of the library is utilized for generation of a library of rAAVs comprising the viral vector on the plasmid and the mutated capsid protein. In some aspects, infection conditions used for rAAV production during library ensure that each production cell (e.g., HEK 293 cell) contains only one version of the cap gene to ascertain that the infectivity of a specific capsid is related to a specific genome.

After infection of a population of cells with the library of rAAVs, single cell RNA sequencing is utilized to identify the profile of infected cells and associated identifier. Genomics is utilized to generate a database where the cellular profile, identifier, and the engineered capsid protein of each rAAV are linked. Accordingly, the identifier, the engineered capsid protein, the cellular profile of each rAAV of the library are known and can be queried to identify capsids that only transduce certain cell-types, in this case microglia (See Section IV herein below).

### III. Nucleic Acid Libraries

The present disclosure also provides a library of nucleic acid constructs encoding a library of rAAVs comprising a plurality of rAAV members. Nucleic acid constructs comprise the AAV vector comprising the identifier sequence, and further comprising a gene encoding the engineered capsid protein. The present disclosure also provides a library of cloning plasmids used to prepare the nucleic acid constructs encoding a library of rAAVs comprising a plurality of rAAV members. The library of rAAVs and cloning plasmids can be as described in Section II herein above.

Any of the nucleic acid constructs described herein are to be considered modular, in that the different components may optionally be distributed among two or more nucleic acid constructs as described herein. The nucleic acid constructs may be DNA or RNA, linear or circular, single-stranded or double-stranded, or any combination thereof. The nucleic acid constructs may be codon optimized for efficient translation into protein, and possibly for transcription into an RNA donor polynucleotide transcript in the cell of interest. Codon optimization programs are available as freeware or from commercial sources.

The nucleic acid constructs can be used to express one or more components of the system for later introduction into a cell to be genetically modified. Alternatively, the nucleic acid constructs can be introduced into the cell to be genetically modified for expression of the components of the system in the cell.

Expression constructs generally comprise DNA coding sequences operably linked to at least one promoter control sequence for expression in a cell of interest. Promoter control sequences may control expression of the engineered capsid protein, the AAV vector, or combinations thereof in bacterial (e.g., E. coli) cells or eukaryotic (e.g., yeast, insect, mammalian, or plant) cells. Suitable bacterial promoters include, without limit, T7 promoters, lac operon promoters, trp promoters, tac promoters (which are hybrids of trp and lac promoters), variations of any of the foregoing, and combinations of any of the foregoing. Non-limiting examples of suitable eukaryotic promoters include constitutive, regulated, or cell- or tissue-specific promoters. Suitable eukaryotic constitutive promoter control sequences include, but are not limited to, cytomegalovirus immediate early promoter (CMV), simian virus (SV40) promoter, adenovirus major late promoter, Rous sarcoma virus (RSV) promoter, mouse mammary tumor virus (MMTV) promoter, phosphoglycerate kinase (PGK) promoter, elongation factor (ED1)-alpha promoter, ubiquitin promoters, actin promoters, tubulin promoters, immunoglobulin promoters, fragments thereof, or combinations of any of the foregoing. Examples of suitable eukaryotic regulated promoter control sequences include, without limit, those regulated by heat shock, metals, steroids, antibiotics, or alcohol. Non-limiting examples of tissue-specific promoters include B29 promoter, CD14 promoter, CD43 promoter, CD45 promoter, CD68 promoter, desmin promoter, elastase-1 promoter, endoglin promoter, fibronectin promoter, Flt-1 promoter, GFAP promoter, GPIIb promoter, ICAM-2 promoter, INF-β promoter, Mb promoter, NphsI promoter, OG-2 promoter, SP-B promoter, SYN1 promoter, and WASP promoter.

Promoters can be constitutive promoters or non-constitutive promoters, including regulated promoters. Promoters can also be tissue-specific, e.g., promoters specific to neural tissue. Any of the promoter sequences may be wild type or may be modified for more efficient or efficacious expression. The DNA coding sequence also may be linked to a polyadenylation signal (e.g., SV40 polyA signal, bovine growth hormone (BGH) polyA signal, etc.) and/or at least one transcriptional termination sequence.

### IV. Computer-Implemented Methods and Systems

The present disclosure also encompasses a computer-implemented method for identifying a rAAV exhibiting preferential tropism to a desired cell type. The method comprises providing or having provided a computerized system comprising a general purpose computer system having at least one processor and computer readable memory storing a database of tropism properties exhibited by a plurality of engineered AAV capsid proteins. Tropism properties include information on the ability of an rAAV of the library to transduce certain cell types, and the inability of the rAAV to transduce remaining cell types. The engineered capsid proteins may be prepared as described in Section II and the tropism properties exhibited by each engineered AAV capsid protein can be determined as described in Section I.

The computerized system further comprises a computer readable medium comprising functional modules including instructions for the general purpose computer which when executed by the at least one processor, cause the at least one processor to query the database and select among the plurality of engineered AAV capsid proteins a capsid protein exhibiting tropism to a desired cell type. The database can further comprise a plurality of cell-type-specific transcriptional profile information associated with each cell type, and a plurality of nucleic acid sequences, each sequence encoding a unique engineered AAV capsid protein. The database can further comprise a plurality of identifier sequences, wherein each identifier is unique to a nucleic acid sequence encoding a unique engineered AAV capsid protein. The tropism properties, the sequence of the identifier, and the cellular profile of each rAAV in the library are linked in the database, and can be queried to identify capsids that only transduce certain cell-types, but fail to transduce other cell types.

In one aspect, the system also comprises an interface unit to display an output of a query. The interface unit may be, for example a display device such as, but not limited to a CRT (cathode ray tube) or LCD (liquid crystal display) monitor. The display device can display information to the user and may include or be in operative communication with an input device such as a keyboard, touchscreen, and/or pointing device (e.g., a mouse or a trackball). An input device may alternatively or in addition, be configured to receive and transmit a signal based on other types of user input, such as voice instruction, or body movement.

It should be understood that the disclosed methods, method steps and/or processor-executable instructions can be implemented or executed by means of any digital electronic system, computer hardware, firmware, software, or any combinations thereof. A processor may take the form of a programmable processor, a computer, or multiple computers, which may be programmed to perform the disclosed methods using any programming language. A program of instructions may comprise a stand-alone program or may have two or more modules, components, subroutines, or the like as known in the art of computer programming. Method steps can be performed by one or more programmable processors executing a computer program to perform functions or aspects of the methods, by operating on input data and generating output information.

A processor may be configured, by way of processor-executable instructions, to receive instructions and data from a memory device, which can be configured for storing instructions and data. A processor, or a computer containing a processor, may be in operative communication with at least one or more mass storage devices for storing data (e.g., magnetic, magneto-optical disks, or optical disks), such that the processor can receive data from or transfer data to such storage device(s). For example, data and/or instruction communications can be performed over a digital communications network.

It should be further understood that the disclosed methods, method steps and/or processor-executable instructions can be performed by a distributed computing system. A distributed computing system includes, for example, a front-end (user-end) interface, middleware, and a back-end, or any combination of two or more of these elements. A front-end component can be, for example, a client computer configured by way of processor-executable instructions to display a graphical user interface through which a user can interact with and provide input to the system. An interface can be embodied in a Web browser interface. A middleware component can be, for example, an application server. A back-end component can be, for example, a data server. Any or all of the components of such a distributed system can be in operative communication by way of one or more digital communications networks, which may be wired and/or wireless networks.

### V. Optimizing Delivery of a Transgene to a Desired Cell Type in a Population

The present disclosure also encompasses a method of optimizing delivery of a transgene to a desired cell type in a population of more than one cell type. The method comprises identifying or having identified an engineered AAV capsid protein exhibiting preferential tropism to the desired cell type. The engineered AAV capsid protein exhibiting preferential tropism to the desired cell type can be identified using a method described in Section I, or can be queried in silico using a system described in Section IV.

The method further comprises transducing a population of cells comprising the desired cell type with an rAAV comprising the identified engineered AAV capsid protein to thereby deliver the transgene to the desired cell type. A cell of the desired target cell type can be a central nervous system cell. In some aspects, the desired target cell type is a microglial cell. In some aspects, the desired target cell type is an astrocyte. The desired cell type can be in a cell culture, an ex vivo tissue, or can be in a subject. For instance, the cell type can be in an aged and diseased primate, in human brain tissue, including post mortem brain/spinal cord tissue kept alive for a few weeks, or from resected tissue from epilepsy patients.

### VI. Kits

The present disclosure also encompasses a kit for identifying or generating engineered AAV capsid proteins exhibiting tropism to a desired target cell type. A kit comprises a library of rAAVs as described in Section II, a library of nucleic acid constructs as described in Section III, or a plurality of cells comprising a library of rAAVs, a library of nucleic acid constructs, or combinations thereof.

The kits may further comprise transfection and transduction reagents, cell growth media, selection media, in vitro transcription reagents, nucleic acid purification reagents, protein purification reagents, buffers, and the like. The kits provided herein generally include instructions for carrying out the methods detailed below. Instructions included in the kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

Methods according to the above can be implemented using computer-executable instructions that are stored or otherwise available from computer readable media. Such instructions can comprise, for example, instructions and data which cause or otherwise configure a general purpose computer, special purpose computer, or special purpose processing device to perform a certain function or group of functions. Portions of computer resources used can be accessible over a network. The computer executable instructions may be, for example, binaries, intermediate format instructions such as assembly language, firmware, or source code. Examples of computer-readable media that may be used to store instructions, information used, and/or information created during methods according to described examples include magnetic or optical disks, flash memory, USB devices provided with non-volatile memory, networked storage devices, and so on.

Devices implementing methods according to these disclosures can comprise hardware, firmware and/or software, and can take any of a variety of form factors. Typical examples of such form factors include laptops, smart phones, small form factor personal computers, personal digital assistants, rackmount devices, standalone devices, and so on. Functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are means for providing the functions described in these disclosures.

As used herein, the term “library” refers to a collection of entities, such as, for example, chimeric capsid proteins, viral particles (e.g., rAAVs), molecules (e.g., nucleic acids), etc. A library may comprise at least two, at least three, at least four, at least five, at least ten, at least 25, at least 50, at least 102, at least 10³, at least 10⁴, at least 10⁵, at least 10⁶, at least 10⁷, at least 10⁸, at least 10⁹, or more different entities (e.g., viral particles, molecules (e.g., nucleic acids)). In some aspects, a library entity (e.g., a viral particle, a nucleic acid) can be associated with or linked to a tag (e.g., a barcode), which can facilitate recovery or identification of the entity. For example, in some aspects, libraries provided herein comprise a collection of rAAVs and libraries of nucleic acid compositions encoding the rAAVs. In some aspects, a library refers to a collection of nucleic acids that are propagatable, e.g., through a process of clonal amplification. Library entities can be stored, maintained or contained separately or as a mixture.

A transgene is a gene that has been transferred naturally, or by any of a number of genetic engineering techniques from one organism to another. The introduction of a transgene, in a process known as transgenesis, has the potential to change the phenotype of an organism. Transgene describes a segment of DNA containing a gene sequence that has been isolated from one organism and is introduced into a different organism. This non-native segment of DNA may either retain the ability to produce RNA or protein in the transgenic organism or alter the normal function of the transgenic organism's genetic code. In general, the DNA is incorporated into the organism's germ line.

As used herein, the term “gene” refers to a segment of DNA that contains all the information for the regulated biosynthesis of an RNA product, including promoters, exons, introns, and other untranslated regions that control expression.

As used herein, “expression” includes but is not limited to one or more of the following: transcription of the gene into precursor mRNA; splicing and other processing of the precursor mRNA to produce mature mRNA; mRNA stability; translation of the mature mRNA into protein (including codon usage and tRNA availability); and glycosylation and/or other modifications of the translation product, if required for proper expression and function.

As used herein, the term “mutant” means any heritable variation from the wild-type that is the result of a mutation, e.g., single nucleotide polymorphism (“SNP”). The term “mutant” is used interchangeably with the terms “marker”, “biomarker”, and “target” throughout the specification.

The terms “polypeptide” and “protein” are used interchangeably to refer to a polymer of amino acid residues.

As used herein, the term “encode” is understood to have its plain and ordinary meaning as used in the biological fields, i.e., specifying a biological sequence. The term “encode,” when used to describe the function of nucleic acid molecules, customarily means to identify one single amino acid sequence that makes up a unique polypeptide, or one nucleic acid sequence that makes up a unique RNA. That function is implemented by the particular nucleotide sequence of each nucleic acid molecule.

As various changes could be made in the above-described cells and methods without departing from the scope of the invention, it is intended that all matter contained in the above description and in the examples given below, shall be interpreted as illustrative and not in a limiting sense.

### EXAMPLES

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The publications discussed throughout are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1. Introduction

The role of microglia in the CNS can be studied using inducible (e.g., tamoxifen-dependent CRE recombinase) transgenic animals. Such approaches lack a certain degree of control. For instance, dosing of the transgene is fixed and constitutive once induced. In contrast, in a regulatable vector system the level of transgene expression can be modulated based on the administration of, for instance, doxycycline. Unfortunately, the use of inducible promoters in a CRE-dependent fashion is not straightforward as such cassettes contain multiple genetic elements and promoters. A CRE system also does not provide simple and efficient control of all types of genetic elements. For instance, small RNAs such as short hairpin RNAs or guide RNAs for CRISPR applications are typically expressed via polymerase (pol) III promoters, and such transcription cassettes cannot be controlled in the same fashion, and the use of pol II promoters have certain limitations in these applications (e.g. imprecise start of transcription). Moreover, this type of manipulation is limited to species where transgenesis is feasible (i.e. mainly mice and rats). However, perhaps the biggest barriers to using transgenic animals to specifically study CRE-mediated genetic manipulations are in situations where genetic manipulations are desired in multiple phenotypically distinct cell types (e.g. microglia and neurons). For instance, the use of the CX3CR1CreER mouse where CRE is expressed specifically in microglia could not be combined with a neuron-specific CRE mouse, as this would ablate any precision of genetic modulation that each animal confers on its own.

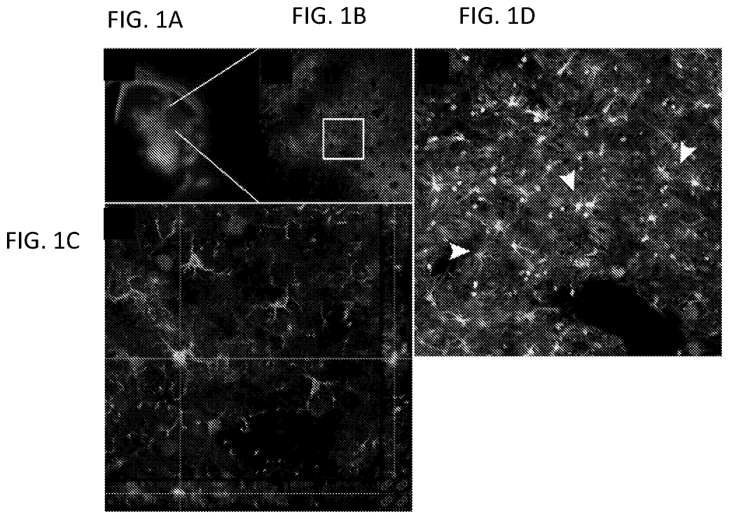

Another modality whereby microglia could be manipulated is viral gene therapy. Nonetheless, lentiviruses (LV) transduce microglia with rather low efficacy in vivo, and adeno-associated viruses (AAV) have proven to be remarkably refractory to microglial transduction. AAV viral vectors that specifically transduce microglia are generated and identified. The inventors have shown (FIG. 1) that there is no biological rationale for this apparent inability of AAVs to effectively target microglia. The AAV capsids utilized in FIG. 1 were generated using “directed” molecular evolution of AAV. This is a popular method whereby AAV variants are selected from large engineered AAV capsid libraries with high diversity, using a positive selection marker. The approach that led up to identification of the capsids utilized in FIG. 1 was based on viral library enhanced with presynaptic ligands, aimed at producing variants with enhanced retrograde transport. Thus far, however, negative selection in the classic sense has not been possible. Therefore, cell specific targeting is not guaranteed (e.g. identified AAV variant that can target microglia, also targets neurons; FIG. 1). To address this technical problem, the inventors provide a novel workflow in which 1) a library of barcoded AAVs targeted to microglia are generated by the incorporation of microglial ligands in to the capsid of AAV2; 2) single cell RNAseq of the transduced mouse brain are used to identify the profile of individual cells transduced by AAVs and the corresponding barcodes; 3) bioinformatics are used as a means to apply “in silico negative selection” against variants (barcodes) that are also present in cell types that are undesirable to target (e.g., allowing us to identify capsids present in microglia but not neurons). This approach builds a large database that delineates the transduction properties of a large number of AAV variants. 4) The utility of this new vector is demonstrated in the context of studying microglial function, and in experimentation that would historically not be possible using classic CRE-based manipulations. In doing so, the investigation is extended to determining if region-specific modulation of microglial epigenetic regulation results in local changes of neuronal excitability of corticostriatal networks. 5) In parallel, experiments expand this approach across species (non-human primates) and across age (aged rodents). Moreover, although the examples herein discuss microglia, the database generated can be queried against barcodes (i.e. capsids) that are specific for subsets of neurons or other non-neuronal cells, providing a large repository of potential cell-specific AAV variants, an extremely powerful tool for the study of the nervous system.

Advantages of AAV over existing technologies to study microglia function in vivo. Manipulating “resting” microglia has proven to be relatively difficult, as these cells readily respond to miniscule changes in their environment. It may thus be difficult to resolve whether the true consequence following a certain manipulation is due to a specific effect on a microglial process, or a generalized response to the changing environment. Nevertheless, the use of transgenic technology such as the CX3CR1 CreER mouse, the current state-of-the-art technology, has allowed a significantly improved resolution as it relates to microglial manipulations, and has provided a crucial backdrop for an improved understanding of microglial function. Yet, the use of such technology, although representing a significant step forward, still has several limitations, most of which can be overcome by the development of a microglia-specific AAV vector. Perhaps the most significant limitation to using a model such as the CX3CR1^{CreER} is that its use still limits the use of intersectional approaches to restrict ectopic expression to molecularly-defined subsets of cells. For instance, this model would not be suited when two or more distinct CRE recombinase-mediated events are needed (e.g. neuron-specific CRE together with microglia-specific CRE). However, with the utility of specific viral vectors, such distinction and precision can be achieved. This utility is demonstrated by combining a microglial specific AAV in animals where neuron-specific CRE expression is labeling neurons and dendrites with YFP. While tamoxifen-based CRE systems allow temporal control of gene expression, it does not provide dose control (i.e. once the recombination event has taken place, gene expression is persistently turned on or off). When used in combination with CRE-dependent AAV vectors (aka “FLEX” vector), the use of various titers allows for some dose control. However, this system lacks the control that can be achieved with a regulatable vector system. For instance, it does not afford a researcher the ability to change the level of expression throughout the course of experimentation. Spatial control of gene expression using CRE expressing transgenics can only be achieved when used in conjunction with CRE-dependent vector systems. On the other hand, the use of ectopically applied CRE-ER (e.g. AAV mediated expression of CRE-ER on a floxed transgenic model) is useful in order to spatially restrict recombination. In both cases, the ability to achieve such manipulations in microglia requires microglial-specific vectors. A minor concern would be potential confounds associated with leakiness of CRE systems, mosaicism, and any toxicity and off-target effects with CRE and/or tamoxifen. Thus, with this in mind, if a single manipulation needs to be made, virally-mediated gene-delivery may add less confound to the study design. One benefit of having the ability to utilize microglia-specific vectors, even for single manipulations, is that it negates the necessity to generate new lines of mice and affords researchers faster and more economical means to gather data. Finally, in the available microglia-specific transgenic mice CX3CR1^{Cre} and CX3CR1^{CreER}, the CRE gene is inserted in place of the CX3CR1 gene, rendering it haploinsufficient for CX3CR1. This, in and of itself, leads to significant changes in plasticity and behavioral phenotypes, making this mouse less than ideal for certain types of experimentation.

Rationale for molecular evolution. Molecular or directed evolution has been tremendously useful in developing novel viral capsids with unique features. However, existing procedures do not allow for a biological process of negative pressure or selection. Rather, often the selection of the terminal capsids identified in these works has been based on extensive testing of numerous variants in order to select for those capsids that produce the lowest degree of a certain off-target activity. In contrast, genomics and bioinformatics are used in order to achieve de facto in silico negative selection.

Moreover, in contrast to previously described methods, a novel method is proposed herein for rational capsid evolution incorporating DNA barcoding. This method keeps all the benefits of rational design while maintaining the broad screening capacity of directed evolution. The novel feature of this method is a viral production approach where each virus particle displays a peptide (from microglial ligands) on the surface which is linked to a unique DNA barcode included in the genome. By design, this method allows for the simultaneous screening of millions of capsid variants in parallel. The screening method only requires a single round of screening, thus the method requires fewer animals and shorter time than existing screening methods.

AAV has remained remarkably refractory to transducing microglia. To date, very little has been reported in terms of strong microglial transduction using AAV. In recent preliminary work using a library approach similar to the one described herein, capsid variants with strong microglial tropism were identified by the inventors (see FIG. 1). Importantly, this finding demonstrates that there is no biological barrier for infecting microglia with AAV. Nevertheless, negative selection in the realm of molecular evolution has not yet been achieved in the study shown in FIG. 1, where the aim was to identify capsids preferentially targeting presynaptic neurons. Accordingly, identified capsids targeted multiple cell-types with high affinity (FIG. 1).

Role of microglia in synapses. Historically, microglia were considered the “garbage-collecting” cell of the CNS, with a pronounced role in phagocytosis of cell debris, misfolded proteins, and clearance of pathogens. However, it is now evident that microglia plays a significant and crucial role in the formation of neuronal connections and networks throughout the lifespan of an organism. During development, microglia modulate neuronal circuitry through phagocytosis of synapses and “unneeded” neurons. This role of microglia persists past development where synaptic pruning is observed in adulthood. This activity occurs in response to a variety of signaling. Indeed, microglia express a variety of receptors for neurotransmitters and other neuromodulators. Thus, neuronal activity is thought to be a key modulator of the role of microglia in synapse and network formation. Importantly, recent evidence demonstrates regional epigenetic differences throughout the brain, differences which modulate microglial activity in the different regions.

Polycomb repressive complex 2 (PRC2) and microglial activity. As eluded to above, microglia exhibit region-specific differences. For instance, striatal microglia have a homeostatic phenotype whereas cerebellar microglia display a clearance phenotype. These phenotypes are epigenetically controlled, where the suppression of the clearance phenotype in striatal microglia is controlled by PRC2. Aberrant deactivation of PRC2 in striatal microglia results in a marked change in the morphology of striatal medium spiny neurons (MSN) and MSN-controlled behavior, the likely result of maladaptive spine pruning, and a reduction in expression of genes that promote spine formation and maintenance. Interestingly, however, PRC2 is also important in neuronal differentiation and function. Silencing of PRC2 in MSN results in the activation of a transcriptional program that results in a neuronal death and neurodegeneration, whereas in other neuronal population drastic changes in dendritic complexity is observed. Thus, modulation of the activity of this complex can have disparate consequences depending on the cell-type targeted.

### Example 2. Guided Molecular Evolution of AAV Paired with Single Cell Bioinformatics and Validation in the Rodent

### Generation of Viral Library.

Viral genome. The wildtype (wt) AAV genome contains two genes: rep (encoding replication proteins) and cap (encoding capsid proteins). The capsid is highly conserved between various natural serotypes, and differs largely in variable regions (VR), areas responsible for cellular receptor binding and subsequent internalization. In AAV2, one such VR is encoded beginning at cap R588, which represents a highly flexible loop which can tolerate insertions of poly-peptides without compromising virion structure or production. FIG. 2 outlines the steps involved in the generation of the barcoded viral genome library. Importantly, insertion at this site disrupts the canonical entry mediated via HSPG binding.

Cloning backbone: FIG. 2 shows a schematic of the cloning system. The plasmid contains key features which include: 1) Cleavage site at R588 to facilitate the insertion of oligonucleotides encoding a short poly-peptide sequence (14 amino acids (AA)), based on microglial ligands (see below). 2) AAV genes are housed outside the iTRs ensuring that serial infectivity cannot occur during virus production. 3) Unidirectional LoxP sites are introduced immediately downstream of cap and immediately upstream of the barcode. Upon CRE-mediated recombination a fragment (containing the upstream iTR) is excised, bringing barcode and ligand insertion sites in close proximity. This facilitates paired-end Illumina sequencing of the barcode together with the ligand sequence in order to build a reference database (1 barcode associated with 1 ligand). RNAseq of the viral genome is done by 4) sequencing of a transcribed mRNA (i.e. GFP) isolated from single nuclei.

Library generation (FIG. 2). The barcoded viral genomes were assembled using standard Gibson cloning methodology (FIG. 2—Step 1). The outlined cloning schema is designed to maximize diversity whilst retaining complete unambiguity (i.e. 1 barcode=1 capsid variant), and to ensure fidelity of barcode/microglial ligand sequencing.

Selection of Microglial Ligands (ML). MLs belonging to two classes were identified: 1) Ligands with known microglial receptors (e.g. LAG-3-associated protein, interferon-γ); and 2) Ligands based on infectious agents that naturally infect microglia. For instance, envelope proteins of human immunodeficiency virus (e.g. HIV-1 YU2, ADA, 89.6, Br20-4, HXB-2 glycoprotein 120) which primarily infect microglia in the CNS. For each identified polypeptide, the library pool consists of oligonucleotides encoding AA 1-14, AA 2-15, AA 3-16, and so on. Oligonucleotides are designed with flanking sequences encoding 5′ and 3′ cap overlap (FIG. 2). Approximately 250 ligands were identified (Table 2). The total number of variants in this library is currently estimated at 130,000.

Barcode generation. Barcodes are generated and inserted as 20 base pair oligonucleotides flanked by a short domain with homology at 5′ and 3′ ends (FIG. 2). Moreover, all barcode oligos are also contained a LoxPTZ17 sequence in the 5 end for CRE recombination. In order to identify the barcode, barcode sequences are based on a cycled (repeated 5 times) V-H-D-B IUPAC ambiguity code in order to allow for maximum variability while avoiding the formation of longer homopolymers.

Viral genome generation. The final genome is generated by Gibson assembly of the 4 components (linearized shuttle vector, microglial targeting sequence, sequence containing C-terminal portion of cap and recombinant AAV genome, and barcode fragment; FIG. 2) in order to ensure a 1:1:1:1 (genome: ML: barcode) ratio. The final ligation product represents a ready-to-package genome (FIG. 2—Steps 1, 4).

Generation of barcode database using paired-end Illumina sequencing. Standard Illumina short-read NGS sequencing does not allow for read lengths that encompass both the barcode and the inserted microglial fragment. In order to prevent this ambiguity, a replica of the original library is utilized. To enable paired-end Illumina sequencing of the library, the plasmid is treated with CRE-recombinase to bring inserted peptide sequence and barcode closer together (FIG. 2—Step 2). In order to prevent recombination between fragments and barcodes, sequencing PCR steps are performed using emulsion PCR. More than 20 million reads are performed to ensure that each variant is sequenced multiple times, ensuring that there is no ambiguity (i.e. the microglial targeting sequence is unequivocally paired with the barcode) with a fidelity approaching 100%. This sequence information is utilized to generate a database where each library genome is identified with one microglial ligand and one barcode.

Viral generation. A key component of viral production during library generation is to ensure that each production cell (i.e. HEK 293 cell) contains only one version of the cap gene, thus allowing the conclusion that the infectivity of a specific capsid is related to a specific genome. Thus, the stoichiometry is different from that of “standard” AAV production. Nevertheless, this methodology still allows for generation of high titer vector preparation (e.g. see FIG. 1 where this packaging method was utilized to isolate capsid variants). Moreover, the separation of the cap gene (FIG. 2—Step 4) from the recombinant genome ensures that released virions cannot cross-infect cells during production. General vector production is performed as described elsewhere. However, given that the canonical receptor binding site is disrupted, column chromatography is performed.

Pilot injections. Viral libraries stemming from the 2 pools of ligand oligonucleotides were unilaterally injected into the striatum (n=3 mice/library) in order to ensure that there is not an overt inflammatory response to the ligands themselves. These animals are analyzed for Iba1 and GFAP immunoreactivity. Comparisons are made between AAV-injected and vehicle-injected hemispheres.

Stereotaxic injections. Vectors (library; n=8 mice (male/female) are injected in to the striatum using standard stereotaxic delivery. The use of a low flow rate (0.5 μl/min, 2 μl total) together with mannitol allows for almost complete transduction of the striatum. This target was chosen because of the ease of which this tissue can be dissected.

Single nuclei isolation is performed using 10× Genomics Chromium scRNA platform. To balance specificity and sensitivity with detection and throughput, ˜7000 nuclei is loaded onto the Chromium instrument in order to recover ˜4000 nuclei with a low 3.1% doublet rate. This process is repeated to achieve a total of ˜20000 nuclei recovered. Power analysis is conducted using 1000 cells to result in 95% power to detect significant differential expression between brain cell types at a false discovery rate of 10%. Finally, this depth allows the identification of inefficient capsids (i.e. capsids that may be present in off-target cells at extremely low numbers due to a general inefficiency of infection).

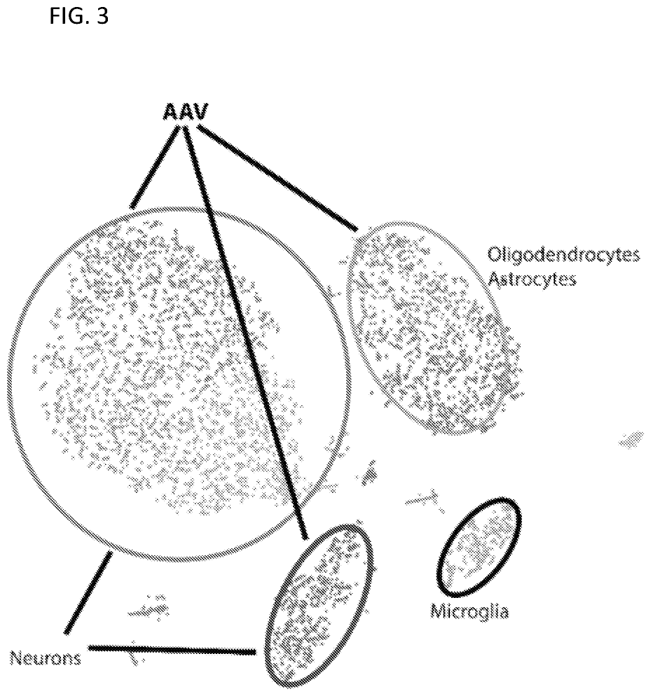

The feasibility of this approach was determined in preliminary studies: In order to determine the ability to 1) Distinguish cell types and 2) Identify rare transduction events, a single striatum was injected with a very low titer of AAV2-CMV-GFP (2 μl of 1×1011 vector genomes/ml). Three days after injection, at a time of very low transgene expression, nuclei were harvested and processed for RNAseq. Using t-Distributed Stochastic Neighbor Embedding (t-SNE), a dimensionality reduction method, both identification of various cell-types in the brain (including microglia which represented roughly 10% of total population), as well as detection of AAV transcripts, were possible (FIG. 3).

However, in the current approach, to ensure capture and enrichment of the viral capsid specific mRNAs, a AAV viral capsid first strand reverse transcriptase primer is doped into the 10× single master mix, based on a similar protocol. To accommodate the inherent 3′ bias of the 10× Chromium system, the primer is designed to the 5′ of the barcode region, and within 100 bp of the polyA tail (FIG. 1). After generation, indexed and pooled single cell libraries is sequenced on an Illumina NovaSeq instrument using a S1 flow cell to a depth of ˜120K reads/cell to provide sufficient information to both determine cell type and to capture low expressing AAV viral capsid barcodes.

Bioinformatics. Once split into barcoded consensus reads, the corresponding microglial ligand sequence is identified using a custom developed workflow incorporating the Frame-Pro, BLAST and HMMER algorithms. The 10×single-cell RNA-sequencing data is processed using Cell Ranger 2.0.2 with a custom built reference. The custom reference consists of the mm10 mouse reference genome plus all unique viral genomes being injected. These viral ligands and their unique barcodes are annotated as genes. Ligand expression can then be directly identified using the standard 10× workflow as previously described. Following the 10× workflow, the cell specific transcriptional profile is used for dimensionality reduction to cluster cell types and qualitatively associate viral ligands to the specific cell types they infect. Moreover, identification of cell types is facilitated with the use of the newly developed Fast Batch Alignment (batch balanced k nearest neighbours-BBKNN). This allows the ability to independently query large-scale mouse scRNA seq data sets, as well as to merge the data from several animals in our approach. Once completed, a database of the matched microglial ligand, barcode, cell specific transcriptional profile, and corresponding cell type is developed.

Importantly, molecular evolution approaches often require multiple rounds of injection, consisting of viral genome isolation, further diversification, and generation of downstream libraries. The approach described herein is not dependent on the isolation of viral genomes, and multiple viral genomes within the same cells do not pose a challenge. Rather, the ability to identify what capsid genes are associated with microglia per se is possible.

### Validation

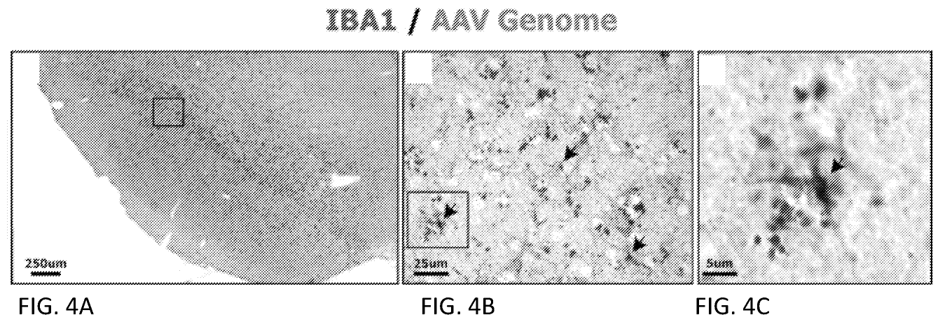

The top six candidates identified using the methodology outlined above are packaged and tested in individual animals (male and female mice, n=5 mice×6 vectors×2 sexes=60 mice) in order to ensure the precision and fidelity of the selected capsids. In this phase of experimentation, relatively high (>10¹³ vector genomes (vg)/ml) titers of AAV is used in order to ensure that lack of non-microglial transduction is not due to dose. Validation consists of 1) Quantitative measurements of transgene (near-infrared densitometry of GFP reporter75), and 2) Qualitative dual label ISH against a non-transcribed portion of the viral genome paired with IHC against microglial markers or neuronal markers (FIG. 4) to ensure fidelity of transduction. 3) Stereology of Iba1+ striatal cells is performed to ensure that there is no microglial toxicity as a result of the vector injection, and near-infrared densitometry of Iba1 and GFAP is performed to confirm the absence of neuroinflammation (normalized against the contralateral hemisphere). 4) Stereology of GFP+microglia. If there is any concern that AAV genomes are present in non-microglial cells, additional animals are generated, and cells are sorted using flow cytometry. Vector genomes thereafter are measured in all different cell populations using our standard digital-droplet PCR bio-distribution protocol.

### AAV Mediated Modulation of PRC2 Activity in Microglia and the Intrinsic Excitability of Local Neurons.

PRC2 is a protein complex with histone methyltransferase activity, and the activity of this complex is crucial for the epigenetic silencing of chromatin. PRC2 in the brain has been associated with cellular differentiation during development. However, this protein has also been ascribed a role in the mature CNS. For instance, in neurons deactivation of this complex can result in drastic changes in dendrite distribution and even neurodegeneration. More importantly, activity of this complex within microglia is associated with region-specific differences in the activity of microglia. As discussed above, activity of PRC2 in striatal microglia facilitates the maintenance of a homeostatic state, whereas disruption of the PRC complex (via removal of the component embryonic ectoderm development protein (EED)), alters the microglial epigenetic and transcriptional profile, and ultimately their function.

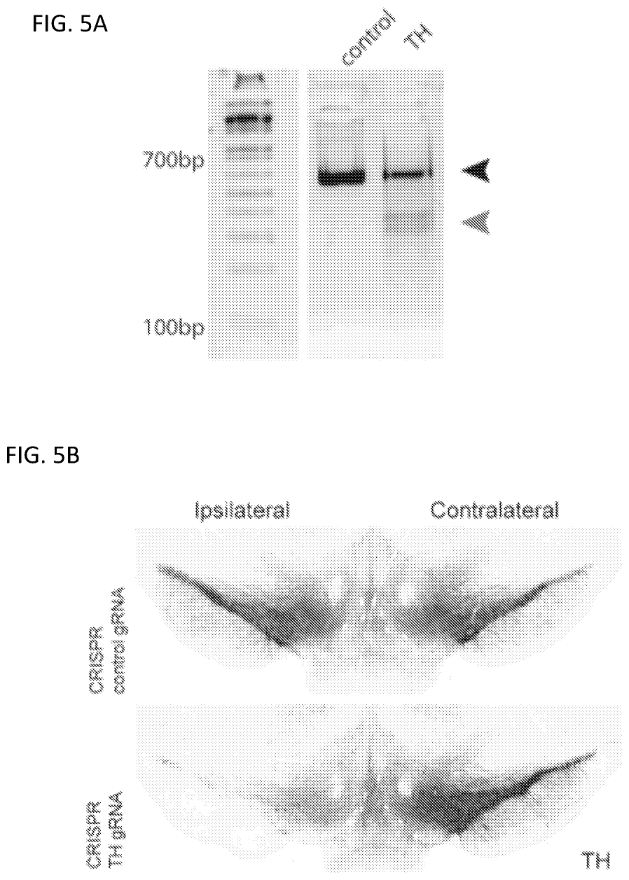

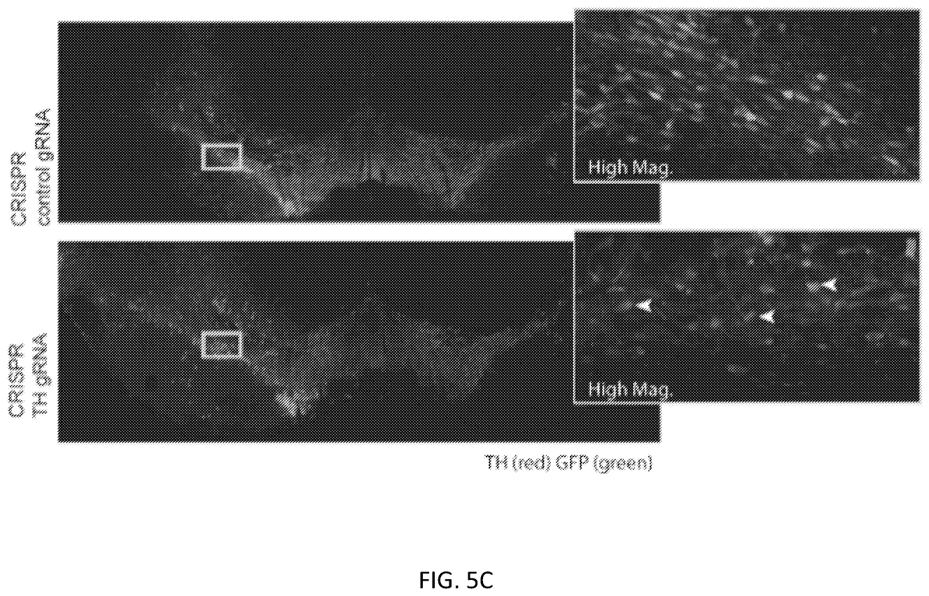

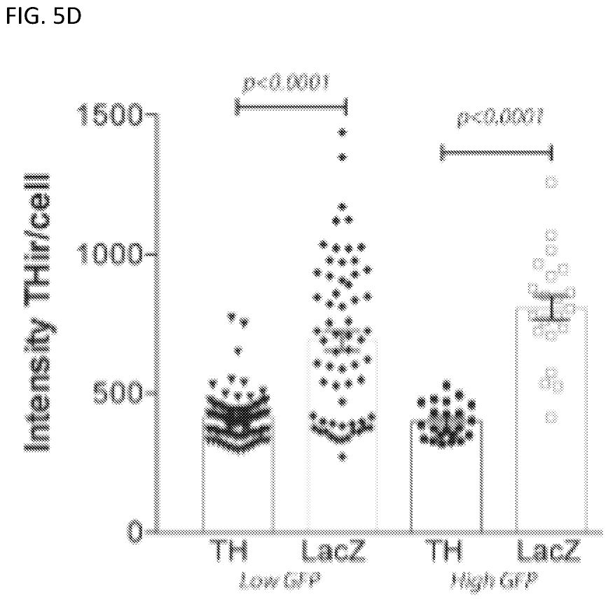

Prior studies have relied upon transgenic mice to specifically modulate microglial transgene expression. However, there are several caveats with this approach. Given the limitations for regions specific control of gene manipulations using the CX3CR1CreER transgenic mouse approach, in the above study, ablation of EED in microglia was performed in the entire brain. Therefore, it is difficult to unequivocally determine if the observed morphological changes in spine density or behavior are caused by changes in local microglia, or if microglial changes in other brain regions affect function or signaling of local afferents, significantly contributing to the phenotype. For instance, changes in cortical neuron firing may have profound effects on MSN morphology and activity. To that end, and as a first validation of the biological utility of the microglial-specific AAV identified herein above (Table 3), the viral vector correlate of this earlier study are performed. However, the analysis is expanded to 1) Include region-specific manipulations, and 2) Perform intracellular electrophysiological recordings to determine the changes in both local and distal neuronal properties as a result of the local changes in microglia. A CRISPR-based approach is used to knockout EED. (see FIG. 5 for feasibility regarding the CRISPR approach). The viral cassette also contains mCherry as a marker of transduction.

This experimentation is performed in transgenic mice where Thy1-mediated CRE expression drives neuronal-specific YFP expression as a means to label neurons and spines (R26R-EYFP mice crossed with B6.Cg-Tg(Syn1-cre)671Jxm/J mice; both are readily available from The Jackson Laboratory). The reason for this cross is multifold: 1) It demonstrates one advantage of utilizing a microglial-specific AAV as opposed to the CX3CR1CreER mouse; as discussed above, it is difficult to combine two or more distinct CRE-dependent cell-type specific tools. However, in the instant approach, it is possible to manipulate 2 distinct cell populations with no chance of overlap. 2) This approach allows the identification and distinguishes transduced microglia that are in close apposition to spines (e.g. “gliapses”). 3) This approach provides a means to specifically label and quantify dendrites and spines.

Approach. CRISPR/Cas has become an immensely popular tool for performing manipulations of gene expression in vitro and in vivo. To that end, an AAV-based cassette was developed to do this type of manipulation in vivo in the CNS. Importantly, given that template-directed editing per se is rather limited in post-mitotic cells, the focus is on the creation of premature stop-codons as an alternative proof-of principle approach. The system was validated by targeting the protein tyrosine hydroxylase (TH). Guide RNAs (gRNAs) were developed to induce insertion-deletions (INDEL) at the 5′ end of the gene in order to facilitate the generation of a null-transcript. As is shown in FIG. 5, this approach is very efficient, leading to the homozygous deletion of TH in a majority of neurons in the targeted area. Here, using the same workflow, gRNAs against the EED subunit of the PRC2 complex is generated. gRNA candidates are tested and validated in mouse NIH/3T3 cells using a mutation detection assay, and the candidate with the highest efficiency of cleavage is cloned into the AAV-CRISPR genome developed by the inventors and packaged into the capsid selected above. A non-sense gRNA expressing cassette is utilized as control.

Validation of the microglial tool: emulation of studies from CX3CR1 mice. Animals receive unilateral stereotaxic injections into either the striatum (2 μl, 0.5 μl/min) or the M1 motor cortex (0.5 μl, 0.25 μl/min100) (n=12 mice (male+female)×2 injection sites×2 vectors×2 outcome measures=96 mice). Four weeks following the vector delivery, animals undergo motor testing (accelerating rotarod and open field testing at both sites). Then, electrophysiological recordings (RFU) and quantitative post-mortem assessment (MSU) are performed.

Rotarod procedure. An accelerating rotarod paradigm (0-100 rpm over 3 minutes) is used. The time latency and speed at the time of fall are recorded.

Open field testing. Locomotive behavior (total ambulatory distance) is measured using automated capture over 10 minutes.

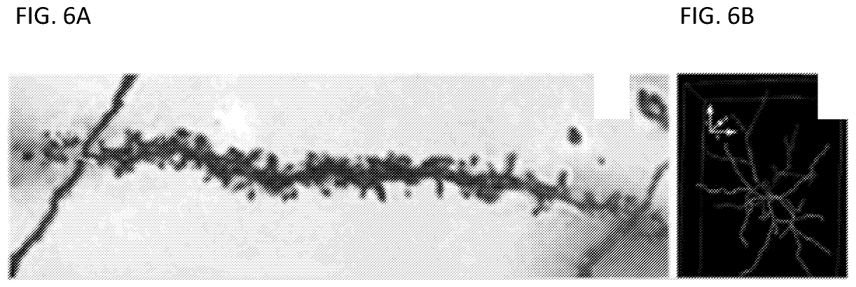

Dendritic Spine Analysis. Striatal and cortical sections are evaluated for changes in spine density and morphology. YFP labeled neurons to be measured is chosen based on the proximity to transduced microglia. Cortical pyramidal dendrites and spines are counted using Neurolucida software. For each animal 20 neurons from each of 2 sections are sampled. Sampling this number of neurons has previously been shown to be sufficient to detect significant differences in spine density in work performed by the inventors involving dyskinetic animals (FIG. 6). For each neuron the dendritic arbors are reconstructed, one dendrite is randomly chosen from each neuron, and the number and phenotype (thin, mushroom, or bifurcated) of spines are quantified in the proximal portion (˜50-90 μm from the cell body) and the distal portion (˜130-170 μm from the cell body). The data is expressed as spines/10 μm.

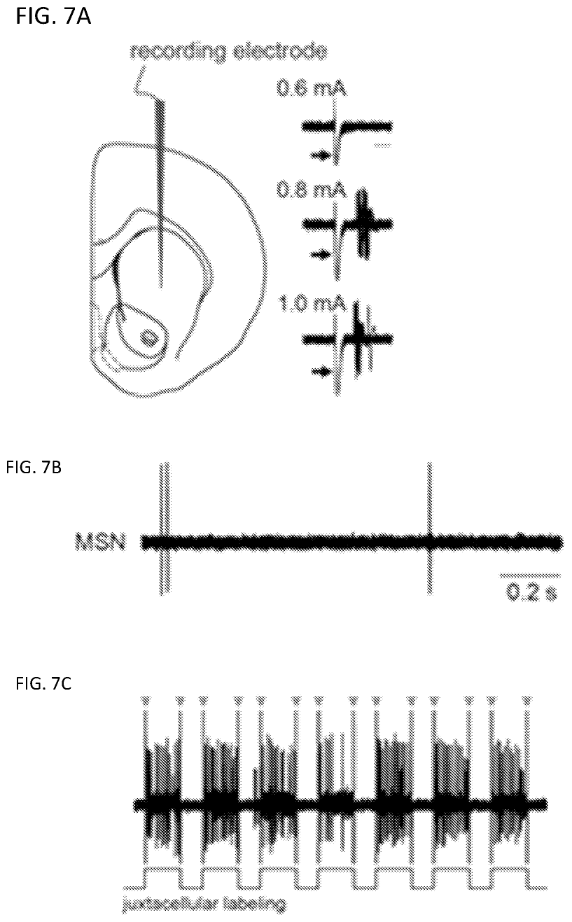

In vivo electrophysiological measurement of neuronal excitability. In vivo extracellular recordings in motor cortex and striatum are used to examine potential changes induced by microglia manipulations in the balance of excitatory and inhibitory transmission. Recordings are made using a NeuroData amplifier. Glass electrodes are pulled using a Narishige (PE-21) electrode puller and filled with: 2 M NaCl (extracellular, impedance: 15-25 MΩ) and 2% neurobiotin for juxtacellular labelling. Electrode potentials are digitized, stored, and analyzed using commercial (Axon) software applications. Concentric bipolar stimulating electrodes (NE100X50) are implanted into the motor cortex ipsilateral to the striatal recording electrode. Spontaneous, afferent-evoked, and antidromic spike activity is measured in the cortex and striatum (see FIG. 7) and compared across the different experimental conditions described above. Based on recent studies by the inventors, it was estimated that 10-12 mice/group are needed to collect 10-15 corticostriatal, striatonigral and striatopallidal neurons. Both males and females were used in the studies and no sex differences were discovered with regard to evoked activity and neuronal firing in WT mice. At the end of the recording session, all neurons are juxtacellularly-labeled and neuronal subpopulations are identified using neurobiotin and IHC double labeling.

Quantitation of knockdown. A quantitative dual ISH/IHC method was previously developed by the inventors, whereby mRNA levels can be quantified in phenotypically distinct cells. The contralateral hemisphere is utilized as a control. Finally, all animals are subject to validation of transduction (i.e. mCherry expression).

It is expected that suppression of microglia function results in a decrease in dendritic complexity in both striatonigral and striatopallidal projection neurons, as well as in cortical pyramidal neurons. As a result, it is expected that knockout of EED and subsequent disruption of the PRC2 complex induces abnormal membrane activity and hyperexcitability in corticostriatal and MSNs as a result of loss of spine density and membrane surface area, an effect which makes these cells more electrotonically compact.

### Example 3. Impact of Species on Efficacy of Microglial Specific Vector Transduction

The top 4 capsids identified in Example 2 are tested in St. Kitts Green Monkeys (Chlorocebus sabaeus). Due to the lack of striatal decussations, it is possible to treat each hemisphere as an independent sample. Each virus is injected into 3 hemispheres (n=3 striata/capsid), thus a total of 6 monkeys are utilized. Subjects receive bilateral injections of rAAV expressing GFP targeted to the putamen. As in the rodent validation studies described above, relatively high titers are utilized to achieve maximum transduction. Importantly, during the same surgical session, each animal also receives an injection of the original library targeted to the cerebellum. This structure is removed from the caudate/putamen, and thus does not influence the outcome of the primary objective of this experiment. Nevertheless, this allows the maximal use of each monkey, and utilization of the cerebellar tissue for single nuclei RNAseq as explained below.

Quantitative postmortem analyses. Tissue is collected at 1 month post-treatment to assess transduction. At euthanasia, subjects are perfused with physiological saline, brains removed, tissue punches collected from caudate nucleus, putamen, the cerebellum is removed, and the remaining tissue immersion fixed in 4% paraformaldehyde. One series of sections is subject to dual ISH/IHC as described above, to assess fidelity of microglial transduction (FIG. 4). Iba1+ immunoreactivity is also measured to identify any inflammation as a result of the vector delivery. Tissue punches are utilized to assess transgene expression using both western blotting and ddPCR. One series is utilized for unbiased stereological estimation of GFP+ cells. Inflammation is assessed using near infrared densitometry of Iba1 and GFAP, and microglial toxicity is assessed using stereology of Iba+ cells. Remaining sections are used as needed. Fresh tissue punches from the caudate/putamen are archived in case cell sorting is needed in order to unequivocally validate fidelity of transduction.

Tissue injected with the viral library and collected from NHPs as described below is used to generate a scRNA seq library as described in Example 1. RNAseq data are collected and processed as described above. Briefly, ˜7000 nuclei are isolated from dissected cerebellar tissue and loaded onto the 10× Genomics Chromium instrument, and indexed and pooled single cell libraries are sequenced on an Illumina NovaSeq instrument (again multiple runs are performed to sequence a sufficient number of microglia). Sequence data are analyzed in the context of the annotated African green transcriptome and the EMBL-EBI atlas, where the bioinformatics approach is exactly as outlined above.

### Example 4. AAV2-GFP Subpial Injection

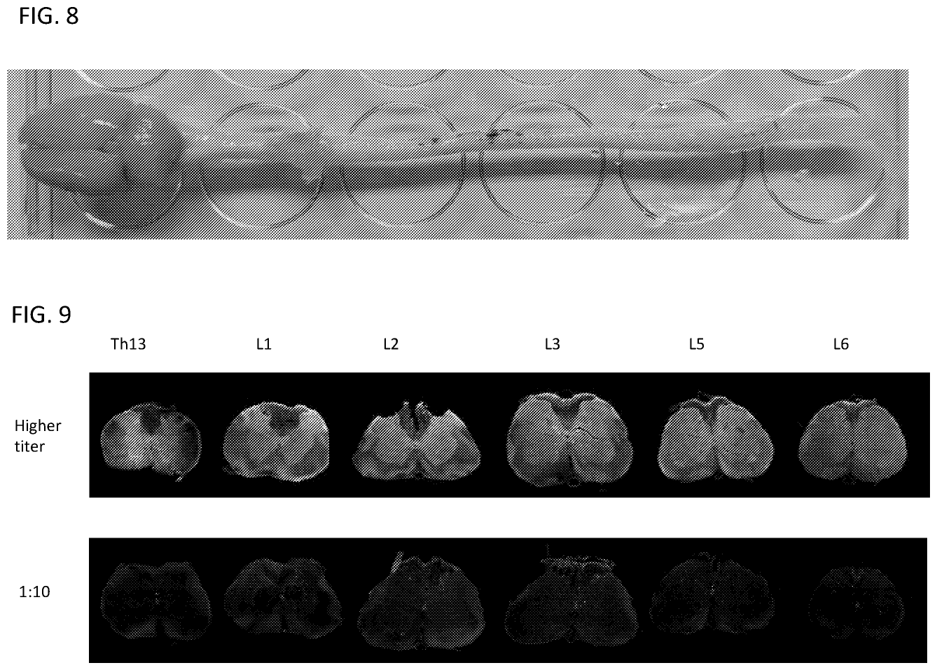

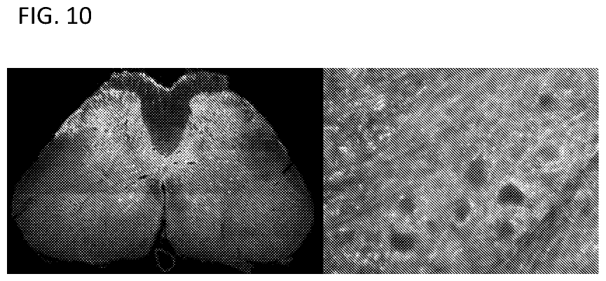

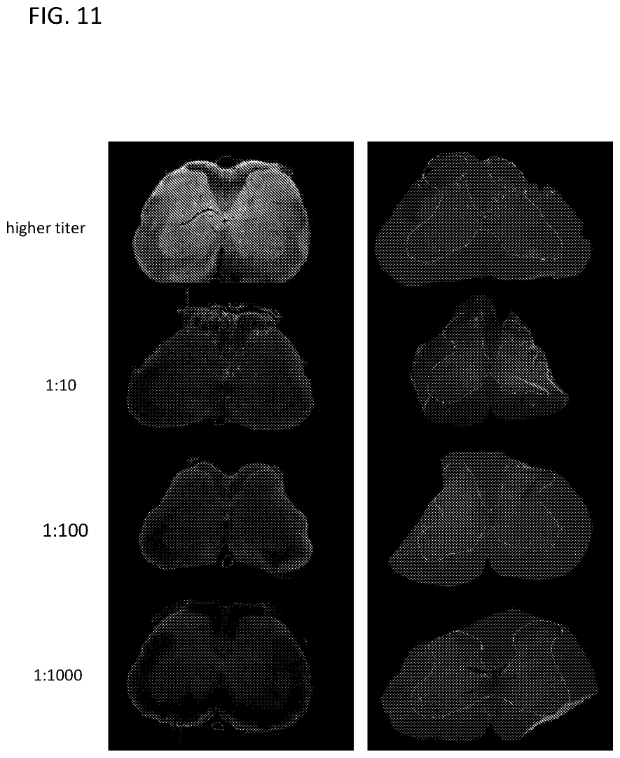

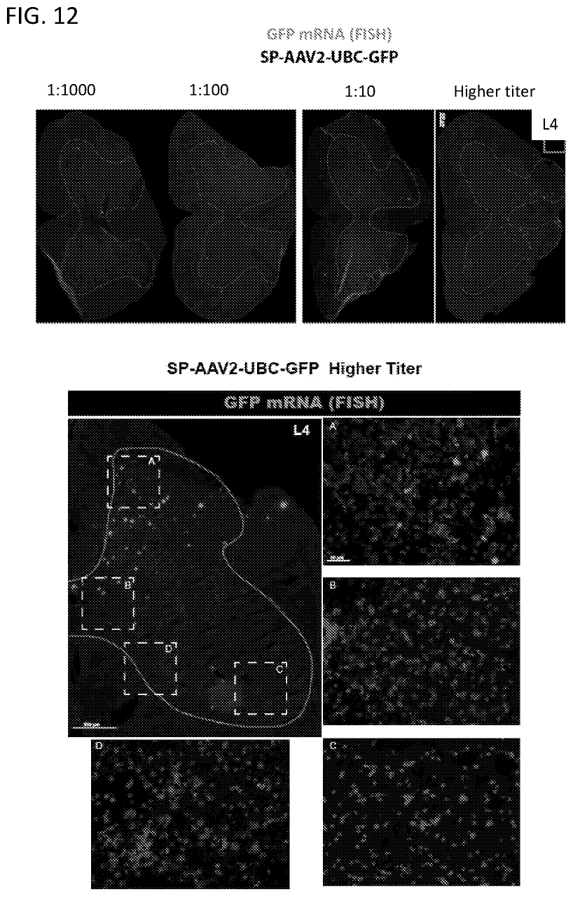

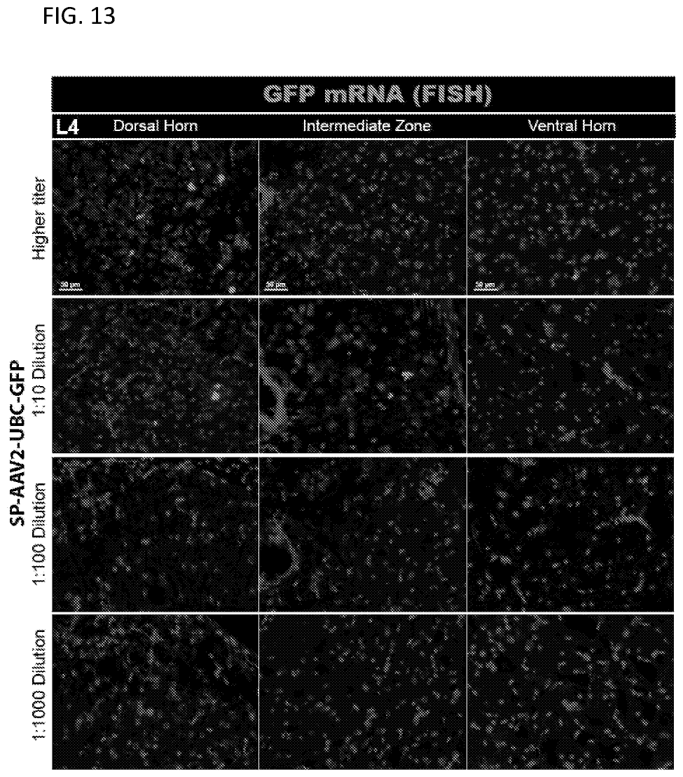

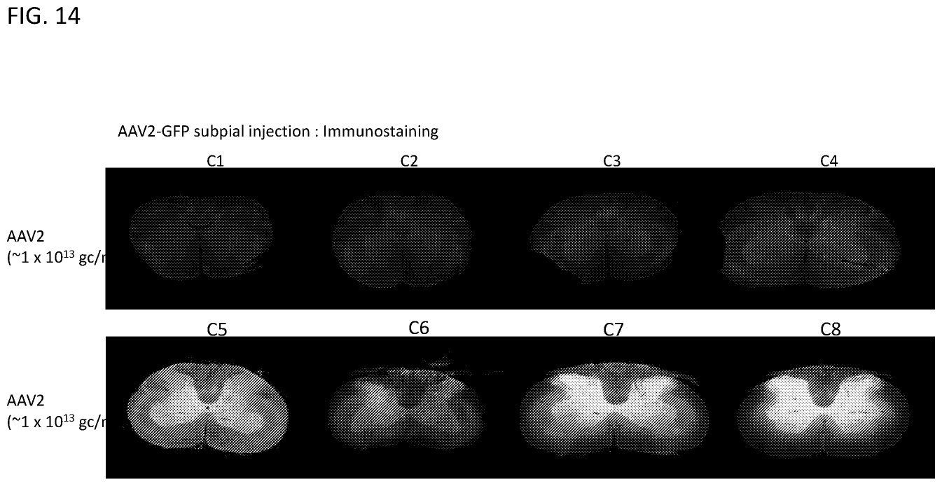

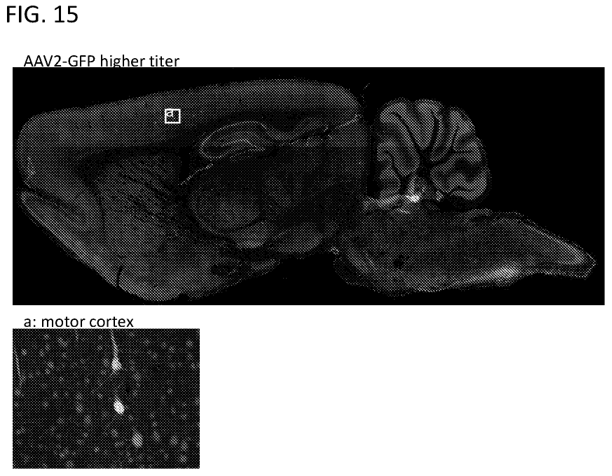

Sprague Dawley rats were injected by subpial injection with ˜1×10¹³ gc/ml of a GFP-expressing rAAV2 having improved neuronal tropism to neurons. One month later the spinal cord was harvested (FIG. 8) and assessed for transduction. Significant transgene (green) expression can be seen throughout thoracic and lumbar sections of the spinal cord (FIGS. 9 to 14). No significant GFP expression can be observed in negative control (spinal cords injected with a lower concentration of the vector). Robust retrograde transduction (i.e. uptake of virus via upper motor neurons and expression of transgene in the brain) can be seen in motor cortex (FIG. 15). Results further show that this virus behaves better in the 1) aged, and 2) deceased nervous system, than common serotypes

What is claimed is:

1. A method for identifying from a population of engineered AAV capsid proteins, a capsid protein exhibiting preferential tropism to a desired cell type, the method comprising:
a. generating a plurality of recombinant AAV virions (rAAVs) each comprising an engineered capsid protein encapsidating an AAV vector, wherein the AAV vector has AAV inverted terminal repeats (ITRs) flanking a transgene and an identifier sequence unique to the capsid protein encapsidating the vector;
b. infecting a population of more than one cell type with the rAAVs of (a) to generate a plurality of transduced cells each comprising an rAAV from (a);
c. determining the sequence of the unique identifier sequence in each transduced cell from (b) to identify the capsid protein present in each cell;
d. determining the cell type of each transduced cell from (b); and
e. identifying a capsid protein exhibiting preferential tropism to the desired cell type based on the presence and absence of the protein in each cell type, wherein the protein exhibits preferential tropism to the desired cell type if the protein is present in the desired cell type and absent in cell types other than the desired cell type.

2. The method of claim 1, further comprising identifying a plurality of engineered AAV capsid proteins, each exhibiting preferential tropism to a desired cell type.

3. The method of claim 1, wherein determining the cell type of each cell comprises determining a transcriptional profile for each cell.

4. The method of claim 1, wherein the transgene encodes a reporter.

5. The method of claim 4, further comprising detecting the transgene in each cell in the population of cells to identify cells transduced with an rAAV.

6. The method of claim 1, wherein a cell of the desired cell type is a neural cell.

7. The method of claim 1, wherein a cell of the desired cell type is a cell of microglial lineage.

8. The method of claim 1, wherein the engineered protein comprises a peptide insertion.

9. The method of claim 8, wherein the peptide insertion is in a region of the capsid protein of AAV2 selected from I-261, I-381, I-447, I-534, I-573, I-587, I-453, I-520, I-588, I-584, I-585, I-588, I-46, I-115, I-120, I-139, I-161, I-312, I-319, I-459, I-496, I-657, Y257, N258, K259, S391, F392, Y393, C394, Y397, F398, Q536, Q539, or a corresponding position in a capsid protein of another AAV serotype.

10. The method of claim 1, wherein the engineered capsid protein is an AAV2 capsid protein comprising the Y444F, Y500F, Y730F, T491V, R585S, R588T, R487G amino acid substitutions, or combinations thereof, or corresponding substitutions in the capsid protein of another AAV serotype.

11. The method of claim 1, wherein the engineered capsid protein is an AAV2 capsid protein comprising the R585S, R588T, and R487G amino acid substitutions, or corresponding substitutions in the capsid protein of another AAV serotype.

12. A computerized system for identifying a rAAV exhibiting preferential tropism to a desired cell type, the computerized system comprising:
a. a general purpose computer having at least one processor;
b. computer readable memory storing a database of tropism properties exhibited by a plurality of engineered AAV capsid proteins identified using a method of claim 1; and
c. a computer readable medium comprising functional modules including instructions for the general purpose computer which when executed by the at least one processor, cause the at least one processor to query the database and select among the plurality of engineered AAV capsid proteins a capsid protein exhibiting preferential tropism to a desired cell type.

13. The computerized system of claim 12, wherein the database further comprises:
a. a plurality of cell-type-specific transcriptional profile information associated with each cell type; and
b. a plurality of nucleic acid sequences, each sequence encoding a unique engineered AAV capsid protein.

14. The computerized system of claim 13, wherein the database further comprises a plurality of identifier sequences, wherein each identifier is unique to a nucleic acid sequence encoding a unique engineered AAV capsid protein.

15. A plurality of recombinant AAV virions (rAAVs), wherein each rAAV member of the plurality of rAAVs comprises an engineered AAV capsid protein encapsidating an AAV vector, wherein the AAV vector has AAV inverted terminal repeats (ITRs) flanking a transgene and an identifier sequence unique to the capsid protein of each rAAV, wherein each engineered AAV capsid protein exhibits preferential tropism to a desired cell type.

16. The rAAV library of claim 15, wherein the engineered capsid protein comprises at least one mutation relative to a wild type capsid protein, and wherein the mutation is selected from a peptide insertion, an amino acid substitution, and an amino acid deletion.

17. The rAAV library of claim 16, wherein the desired cell type is a glial cell.

18. The rAAV library of claim 17, wherein each peptide insertion is derived from an amino acid sequence of SEQ ID NO 2-183.

19. The rAAV library of claim 15, wherein each rAAV exhibits preferential tropism to a desired target cell type.

20. A plurality of nucleic acid constructs encoding the plurality of rAAVs of claim 15.

21. A plurality of cells comprising the plurality of rAAVs of claim 15, the plurality of nucleic acid constructs encoding the plurality of rAAVs of claim 20, or a combination thereof.

22. A method of optimizing delivery of a transgene to a desired cell type in a population of more than one cell type, the method comprising:
a. identifying or having identified an engineered AAV capsid protein exhibiting preferential tropism to the desired cell type by the method of claim 1 or by the computerized system of claim 12; and
b. transducing a population of cells comprising the desired cell type with an rAAV comprising the engineered AAV capsid protein identified in (a) to thereby deliver the transgene to the desired cell type.

23. The method of claim 22, wherein a cell of the desired target cell type is a central nervous system cell.

24. The method of claim 23, wherein a cell of the desired target cell type is a microglial cell or an astrocyte.

25. A kit for identifying or generating engineered AAV capsid proteins exhibiting preferential tropism to a desired target cell type, the kit comprising a library of rAAVs of claim 15, a library of nucleic acid constructs of claim 20, or a plurality of cells of claim 21.
